(19) United States
(12) Patent Application Publication
(10) Pub. No.: US 2015/0150519 A1
(43) Pub. Date: Jun. 4, 2015

(54) APPARATUS AND METHODS FOR MANAGING BLOOD PRESSURE VITAL SIGN CONTENT IN AN ELECTRONIC ANESTHESIA RECORD.

(71) Applicant: David Michael Glenn, Menlo Park, CA (US)

(72) Inventor: David Michael Glenn, Menlo Park, CA (US)

(73) Assignee: David Michael Glenn, Menlo Park, CA (US)

(21) Appl. No.: 14/513,355

(22) Filed: Oct. 14, 2014

Related U.S. Application Data
(60) Provisional application No. 61/896,109, filed on Oct. 27, 2013.

(51) Int. Cl. A61B 5/00; G06F 3/0488; G06F 3/0484; A61B 5/021; G06F 3/0481

(52) U.S. Cl. CPC A61B 5/748; A61B 5/021; A61B 5/4821; A61B 5/7435; G06F 3/0481; G06F 3/04847; G06F 3/04845; G06F 3/04883

(57) ABSTRACT

A graphical user interface and methods for managing blood pressure vital sign content in an electronic anesthesia record on a multi-function gesture-sensitive device via gesture-based means. The graphical user interface and methods give an improved user interface and set of functions via novel functions unique to gesture sensitive interfaces.

### CROSS REFERENCE TO RELATED APPLICATIONS

This application claims priority under 35 U.S.C. §119 (e) to U.S. provisional patent application Ser. No. 61/896,109 filed on Oct. 27, 2013, the contents of which is hereby incorporated herein by reference in its entirety for all purposes.

### COPYRIGHT NOTICE

Pursuant to 37 C.F.R. 1.71(e), applicants note that a portion of this disclosure contains material that is subject to and for which is claimed copyright protection, such as, but not limited to, copies of paper forms, screen shots, user interfaces, electronic medical record formats, or any other aspects of this submission for which copyright protection is or may be available in any jurisdiction. The copyright owner has no objection to the facsimile reproduction by anyone or the patent document or patent disclosure, as it appears in the Patent Office patent file or records. All other rights are reserved, and all other reproduction, distribution, creation of derivative works based on the contents, public display, and public performance of the application or any part thereof are prohibited by applicable copyright law.

### STATEMENT REGARDING FEDERALLY SPONSORED RESEARCH OR DEVELOPMENT

None.

### THE NAMES OF PARTIES TO A JOINT RESEARCH AGREEMENT

None.

### FIELD OF INVENTION

This invention relates to an improved technique for managing blood pressure vital sign content via gesture-based means in an electronic anesthesia record.

### BACKGROUND OF THE INVENTION

The introduction of electronic anesthesia record systems was attempted as early as in 1979, although their efficient application has become reality only in the past few years. The advantages of the electronic interface over paper are apparent: reduction of paper, electronic storage of records, graphical user interfaces, consolidation of functions, analytics and trending, ease of use and quick functional updates. Given the complexity of even routine physiological monitoring in contemporary surgical practice, digital systems offer the ability to collect the large volume of perioperative patient information needed for surgical and critically ill patients. An AIMS automatically creates a clinical anesthesia record, generates specialized patient-specific reports for clinical care and billing, and builds an electronic database and searchable repository of physiological and demographic data. An AIMS also makes it possible for anesthesiologists and institutions to meet the increasing demands for legible, comprehensive, secure, and shareable perioperative clinical documents. Despite these challenges, many institutions still rely on paper-based solutions in spite of the electronic advancements of the past two decades. Adoption remains a paramount challenge.

At the heart of this adoption problem is human computer interfacing. Many AIMS systems present significant human-computer functional and usability problems which burden the anesthesiologist while in the operating room. In that setting, the anesthesiologist is required to operate machinery that delivers medications to the patient (such as a mask and a respiration bag), monitor the patients vital signs, keep his/her eyes on the patient to carefully monitor outward physiological states, pay attention and participate in the actions and conversations between doctors and nurses. In addition to basic measurements, such as pulse, blood pressure, and temperature, anesthesiologists also measure the patient's respiration. If the patient is under a general anesthetic, the anesthesiologist measures the volume the patient inhales and the carbon dioxide level exhaled. During some procedures, the anesthesiologist must monitor the volume of blood pumped by the heart, nerve functions or the blood pressure inside the patient's lungs. If the procedure requires the use of special monitors, such as arterial catheters, the anesthesiologist is typically responsible for placing them. Anesthesiologists also ensure that patients remain in the proper position, such as keeping the patient's head aligned during neck surgery.

Many prior art AIMS require the anesthesiologist to use both hands to input data during the procedure. This is because they rely on both a keyboard and mouse or some inefficient combination there of. Some AIMS do enable a form of touch sensitivity, however, their reliance on standard UI controls limit the user experience. In one example of the prior art, when the anesthesiologist wants to enter a medication dosage, he touches the screen and the application pops up a text entry field where he must type a value with a physical keyboard. In another example, the anesthesiologist will touch the screen and to change the default dosage he must use a mouse or tap a scroller on a combo box or other UI control to arrive at the desired value. Many prior art systems rely on a hybrid interfacing means of mouse and keyboard and touch screen, leaving with anesthesiologist with a difficult experience. Worse still, some prior art solutions also require significant space for the monitor, CPU, keyboard holder and mouse pad, making their placement in the crowded operating room cumbersome for all medical personal. This is made more difficult by the fact that the anesthesiologist is often required to input data on many types of medications being administered, many different vital signs being monitored, and many different types of blood pressures being tracked. To help deal with this deluge of real-time information, most prior art AIMS collect data directly and sometimes exclusively through machine interfaces to the equipment present in the operating room.

In this approach, the machine interface may produce a large amount of artifactual data that is irrelevant or spurious. This information, if recorded into a permanent record, may convey a false picture of the medical facts present in the case. Additionally, doctors prefer the ability to add, edit, or delete values based on their perception and clinical interpretation of the data and at a time of their choosing and when it is clinically appropriate and safest for patient care to do so. In some prior art AIMS the anesthesiologist is only presented with a read-only interface to the data being collected. No editing is permitted. Some of these have gone so far as to present a web based, read-only interface formatted to look appropriate for a tablet interface.

Finally, irrespective of the approach, many AIMS simply lack functions often desired but difficult to achieve with conventional interfacing. They include:
Complete access and control of AIMS features via one hand event without a mouse.
A tablet based form factor.
An ability to input multiple medications, with individual types distinguished by color with the ability to add, edit, undo and delete values via a single hand gesture
An ability to store and retrieve medications based on a template.
The ability to visually track time related to multiple events of the case.

Recent advances in tablets and gesture control driven multi-functional devices has enabled another radical advancement in the usability of AIMS, much of which still remains unexplored by the prior art.

### SUMMARY OF THE INVENTION

The invention focuses on the problem of managing blood pressure and heart rate vital signs in an electronic anesthesia record through gesture based means on a multi-function gesture-sensitive device. It is a graphical user interface apparatus and a plurality of embodied methods whose functions are both novel and uniquely implemented by single-hand, gesture based interfacing techniques not heretofore considered in an electronic anesthesia record. In one embodiment, an application program displays a graphical user interface apparatus, which provides the critical interfacing for performing the claimed process embodiments herein.

The objectives of the embodied processes are to allow for the following functions related to blood pressure and heart rate management; individual entry of values, mass entry of values, mass-edit of values, inspect, delete, scale-shift, association to case notes, snap-to-grid, time-shifting and zoom and magnification.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the invention. The invention is better understood by reference to one or more of these drawings in combination with the detailed description of specific embodiments presented herein.

FIG. 1 is the graphical user interface for an electronic anesthesia record on a multi-function gesture-sensitive device and the components of the GUI apparatus for managing blood pressure vital sign content.

FIG. 2 is a visualization of a type 1 blood pressure value using a representative geometric object to display the value.

FIG. 3 is a visualization of a type 2 blood pressure value using two representative geometric objects to display the values for systolic and diastolic pressure.

FIG. 4 is a visualization of a type 3 blood pressure value using three representative geometric objects to display the values for systolic and diastolic pressure and heart rate.

FIG. 5 depicts the relationship between integers in the first sub-view and the grid lines in the second sub-view.

FIG. 6 depicts the relationship between the blood pressure vital sign grid and the second sub-view.

FIG. 7 shows the sub-view of the GUI apparatus for configuring blood pressure and heart rate vital signs.

FIG. 8 shows the GUI apparatus for managing blood pressure and heart rate vital signs and depicts a means of selecting between blood pressures under management.

FIG. 9A-FIG. 9D shows the embodiment of the invention allowing for the mass input of blood pressure and heart rate values.

FIG. 9A shows the GUI apparatus for managing blood pressure and heart rate vital signs and depicts the anesthesiologist initiating the mass input of Type 3 systolic blood pressure values on a multi-function gesture-sensitive device in accordance with an embodiment of the invention.

FIG. 9B shows the GUI apparatus for managing blood pressure and heart rate vital signs and depicts the anesthesiologist inputting a multitude of Type 3 systolic blood pressure values on a multi-function gesture-sensitive device in accordance with an embodiment of the invention.

FIG. 9C shows the GUI apparatus for managing blood pressure and heart rate vital signs and depicts the anesthesiologist inputting a multitude of Type 3 diastolic blood pressure values on a multi-function gesture-sensitive device in accordance with an embodiment of the invention.

FIG. 9D shows the GUI apparatus for managing blood pressure and heart rate vital signs and depicts the anesthesiologist inputting a multitude of Type 3 heart rate values on a multi-function gesture-sensitive device in accordance with an embodiment of the invention.

FIG. 10A-FIG. 10D shows the embodiment of entering one blood pressure and heart rate value at a time.

FIG. 10A shows the first step in the process for entering one blood pressure and heart rate value at a time.

FIG. 10B shows the second step in the process for entering one blood pressure and heart rate value at a time.

FIG. 10C shows the effect of entering a diastolic value greater than the systolic value.

FIG. 10D shows the third step in the process for entering one blood pressure and heart rate value at a time.

FIG. 11A-FIG. 11F shows the embodiment for the mass editing of blood pressure and heart rate values.

FIG. 11A shows the first step in a process for mass editing of blood pressure and heart rate values.

FIG. 11B shows the background color change that occurs when the apparatus going into edit mode.

FIG. 11C shows the anesthesiologist performing the gesture to determine which blood pressure and heart rate values will be part of the mass edit.

FIG. 11D shows which blood pressure and heart rate values are part of the mass edit.

FIG. 11E shows the anesthesiologist performing the mass edit.

FIG. 11F shows the result of performing the mass edit.

FIG. 12A-FIG. 12D shows the embodiment for inspecting blood pressure and heart rate numerical values.

FIG. 12A shows the GUI apparatus and depicts the anesthesiologist inspecting blood pressure and heart rate numerical values on a multi-function gesture-sensitive device in accordance with an embodiment of the invention.

FIG. 12B shows the GUI apparatus and depicts the effect of inspecting blood pressure and heart rate numerical values on a multi-function gesture-sensitive device in accordance with an embodiment of the invention.

FIG. 12C shows the GUI apparatus and depicts the anesthesiologist de-inspecting blood pressure and heart rate numerical values on a multi-function gesture-sensitive device in accordance with an embodiment of the invention.

FIG. 12D shows the GUI apparatus and depicts the de-inspected blood pressure and heart rate values.

FIG. 13A-FIG. 13C shows the embodiment for scale-shifting shifting to enhance the display of blood pressure and heart rate values.

FIG. 13A shows the GUI apparatus and depicts the anesthesiologist scale-shifting to enhance the display of blood pressure and heart rate values on a multi-function gesture-sensitive device in accordance with an embodiment of the invention.

FIG. 13B shows the GUI apparatus and depicts the effect of the scale-shift on blood pressure and heart rate values on a multi-function gesture-sensitive device in accordance with an embodiment of the invention.

FIG. 13C shows the GUI apparatus and depicts the background color change in the scale-shifted area on the second sub-view indicating the area was shifted.

FIG. 14A-FIG. 14C shows the embodiment for the anesthesiologist initiating the process for deleting a plurality of blood pressure and heart rate values.

FIG. 14A shows the anesthesiologist initiating the process for deleting a plurality of blood pressure and heart rate values on a multi-function gesture-sensitive device in accordance with an embodiment of the invention.

FIG. 14B shows the second sub-view going into delete mode and a change in the background color on a multi-function gesture-sensitive device in accordance with an embodiment of the invention.

FIG. 14C shows the anesthesiologist performing a delete of blood pressure and heart rate values on a multi-function gesture-sensitive device in accordance with an embodiment of the invention.

FIG. 15A-FIG. 15D shows the embodiment for representing case notes.

FIG. 15A shows a representative case note along with the case note editor on a multi-function gesture-sensitive device in accordance with an embodiment of the invention.

FIG. 15B shows the anesthesiologist adding a case note by first adding the note to the second sub-view on a multi-function gesture-sensitive device in accordance with an embodiment of the invention.

FIG. 15C shows a plurality of case notes along with the case note editor on a multi-function gesture-sensitive device in accordance with an embodiment of the invention.

FIG. 15D shows the anesthesiologist adding a case note by first adding the note to the case note editor on a multi-function gesture-sensitive device in accordance with an embodiment of the invention.

FIG. 16A-FIG. 16B shows the embodiment for performing the snap-to-grip enablement.

FIG. 16A shows the effect in the second sub-view of the snap-to-grid function on a multi-function gesture-sensitive device in accordance with an embodiment of the invention.

FIG. 16B shows the snap-to-grid button on a multi-function gesture-sensitive device in accordance with an embodiment of the invention.

FIG. 17A-FIG. 17G shows the embodiment for time-shifting the vital sign grid.

FIG. 17A shows an area of the blood pressure vital sign grid that has clustered values and requires a time-shift to better visualize.

FIG. 17B shows an area of the blood pressure vital sign grid that was time-shifted for better visualization.

FIG. 17C shows the effect of multiple time-shifted regions of the blood pressure vital sign grid.

FIG. 17D shows an area of the blood pressure vital sign grid where the application program has changed the background color indicating a time-shift has occurred.

FIG. 17E shows the effect of changing the time resolution of the blood pressure vital sign grid.

FIG. 17F shows the mechanics of show how the application program determines which grid lines to time-shift on the blood pressure vital sign grid.

FIG. 17G shows the effect of changing the time resolution of the blood pressure vital sign grid.

FIG. 18A-FIG. 18B shows the embodiment for zooming and magnifying regions of the display.

FIG. 18A shows the gesture sensitive regions, which can be zoomed and expanded.

FIG. 18B shows the before and after effect of zooming and magnifying.

FIG. 19A-FIG. 19B shows the embodiment for scrolling to the right and left.

FIG. 19A shows the apparatus scrolling to the right.

FIG. 19B shows the apparatus scrolling to the left.

FIG. 20A-FIG. 20R shows the embodiment for undoing and redoing blood pressure content edits.

FIG. 20A shows the anesthesiologist adding the systolic part of a blood pressure value.

FIG. 20B shows the anesthesiologist adding the diastolic part of a blood pressure value.

FIG. 20C shows the anesthesiologist adding the heart rate part of a blood pressure value.

FIG. 20D shows the anesthesiologist adding the systolic part of a blood pressure value.

FIG. 20E shows the anesthesiologist adding the diastolic part of a blood pressure value.

FIG. 20F shows the anesthesiologist adding the heart rate part of a blood pressure value.

FIG. 20G shows the anesthesiologist undoing the heart rate part of a blood pressure value.

FIG. 20H shows the anesthesiologist undoing the diastolic part of a blood pressure value.

FIG. 20I shows the anesthesiologist undoing the systolic part of a blood pressure value.

FIG. 20J shows the anesthesiologist undoing the heart rate part of a blood pressure value.

FIG. 20K shows the anesthesiologist undoing the diastolic part of a blood pressure value.

FIG. 20L shows the anesthesiologist undoing the systolic part of a blood pressure value.

FIG. 20M shows the anesthesiologist redoing the systolic part of a blood pressure value.

FIG. 20N shows the anesthesiologist redoing the diastolic part of a blood pressure value.

FIG. 20O shows the anesthesiologist redoing the heart rate part of a blood pressure value.

FIG. 20P shows the anesthesiologist redoing the systolic part of a blood pressure value.

FIG. 20Q shows the anesthesiologist redoing the diastolic part of a blood pressure value.

FIG. 20R shows the anesthesiologist redoing the heart rate part of a blood pressure value.

FIG. 21A-FIG. 21D shows the embodiment for changing the presentation scheme.

FIG. 21A shows the invention in an alternate presentation scheme.

FIG. 21B shows the invention in an alternate presentation scheme.

FIG. 21C shows the invention in an alternate presentation scheme.

FIG. 21D shows the invention in an alternate presentation scheme.

### DETAILED DESCRIPTION OF THE INVENTION

The above deficiencies and other problems associated with managing blood pressure vital sign information in an electronic anesthesia record are reduced or eliminated by the disclosed apparatus and methods. In all embodiments, the device has a gesture-sensitive display with a graphical user interface (GUI) produced by an application program operating on the portable multi-function device, one or more processors, memory and one or more modules, programs or sets of instructions stored in the memory for performing multiple functions. In some embodiments, the user interacts with the GUI primarily through touch gestures such as one or more fingers directly contacting the gesture-sensitive interface, however other means may also include, but not limited to, kinetic motion gestures or even audio command, sounds or phrases, all of which are considered gesture enablers and hence equivalent. Instructions for performing these functions may be included in a computer program product configured for execution by one or more processors.

It shall be understood that, although the terms first, second, etc. used herein to describe various elements, these elements should not be limited to these terms. These terms are only used to distinguish one element from another. The terminology used in the description of the invention herein is for the purpose of describing particular embodiments only and is not intended to be limiting of the invention.

The word “manage” shall be construed to comprise the functions of display, add, edit, delete, organize, and inspect of blood pressure vital sign information and the graphical user interface effects that enable those functions within the context of an electronic anesthesia record.

As used in the description of the invention and the appended claims, the singular forms “a”, “an” and “the” are intended to include the plural forms as well, unless the context clearly indicates otherwise. The term “plurality” is taken to mean zero to many occurrences.

It will also be understood that the term “and/or” as used herein refers to and encompasses any/all possible combinations of one or more of the associated listed items.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The use of particular gestures is representative only and the reliance of touch-sensitivity is not a requirement as other means of enabling gestures may presently or in the future be possible. It is not implied that the use of the word “gesture” excludes the possibility of a combination of other gestures even of varying types.

The invention does not rely on any particular implementation of a multi-functional, gesture-sensitive device or any version of its operating system and any graphics displayed is not intended to convey a reliance on any particular vendor or version thereof. It shall be understood, that a person of ordinary skill in the art, if given a stock operating system could implement the various embodiments of the invention and the stock operating system has not been modified in any way by the invention, sometimes referred to as “hacks”, “jailbreaks”, “rooting” or “privilege escalations” to name a few.

It should be noted that the use of terminology such as window, sub-window, view and sub-view are representative of a concept in graphical user interface programming and are not limited in any way to one particular vendors approach. Some vendor's application programming interfaces (API) use terms like window/sub-window or panel/sub-panel. All are equivalent and exchangeable herein and convey a logical rather than a physical function, which a person of ordinary skill in programming a particular vendor's API could implement without undue experimentation.

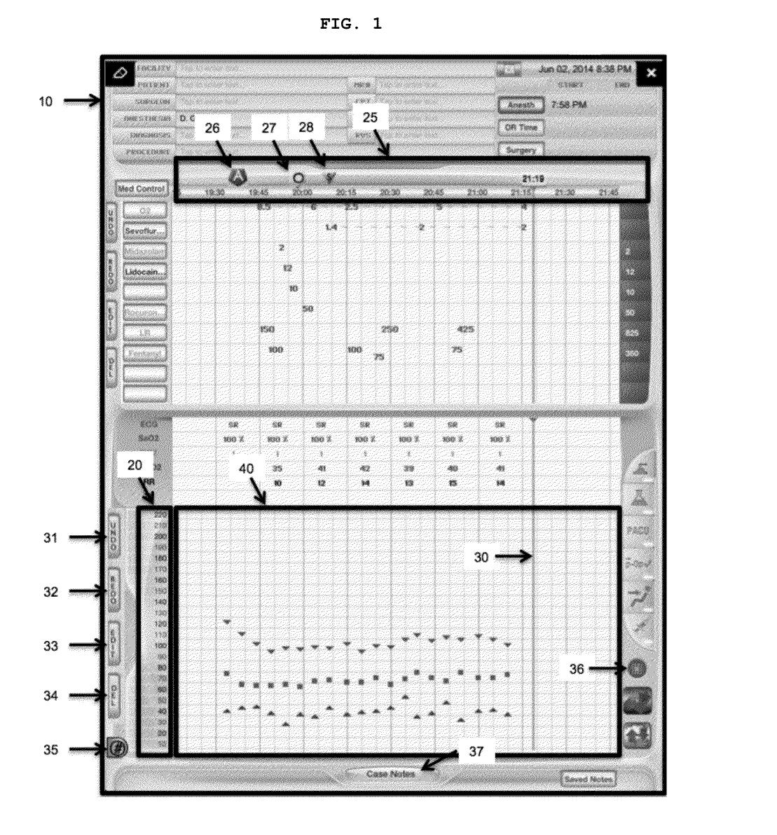

The electronic anesthesia record (FIG. 1) is both an apparatus and a plurality of computer-implemented methods used in conjunction with a portable multifunction device with a gesture-sensitive display and reduced to practice in the form of an application program. The application program displays an application program window (10).

In an embodiment of the invention, the application program operatively enables, via gesture-based means, a graphical user interface apparatus for managing blood pressure vital sign content, shown within the application program window (FIG. 1, 10). The apparatus contains a first, second, third, fourth and fifth sub-view.

The first sub-view displays a vertical set of integers (FIG. 1, 20) representing a dual-purpose integer scale of blood pressure in mmHg and heart rate in beats per minute, ranging from low integers on the bottom to high integers on the top.

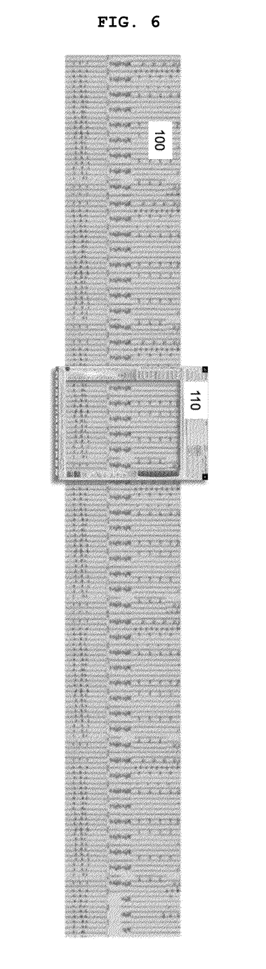

The second sub-view (FIG. 1, 40 and FIG. 6, 110) displays at least a portion of the blood pressure vital sign grid (FIG. 6, 100) and whose purpose is to provide a user interface for managing blood pressure vital sign content. The grid is comprised of rows and columns. The rows representing blood pressure or heart rate values, the columns representing time increments advancing forward in time from left to right. The blood pressure vital sign grid is a record of all blood pressure and heart rate values collected over the life of the electronic anesthesia record. The rows displayed in the second sub-view are aligned horizontally to the integers displayed in the first sub-view such that they identify the blood pressure or heart rate value for a given row line below the integer (FIG. 5). The application program enables the second sub-view to horizontally scrollable back (in time) and forward (to current time or the last recorded blood pressure or heart rate value) via a gesture (two finger horizontal slide). The second sub-view has five operational modes: default, mass-add, edit, delete, and inspect.

The application program enables the anesthesiologist to perform the following functions within the second sub-view and in cooperation with the other sub-views.

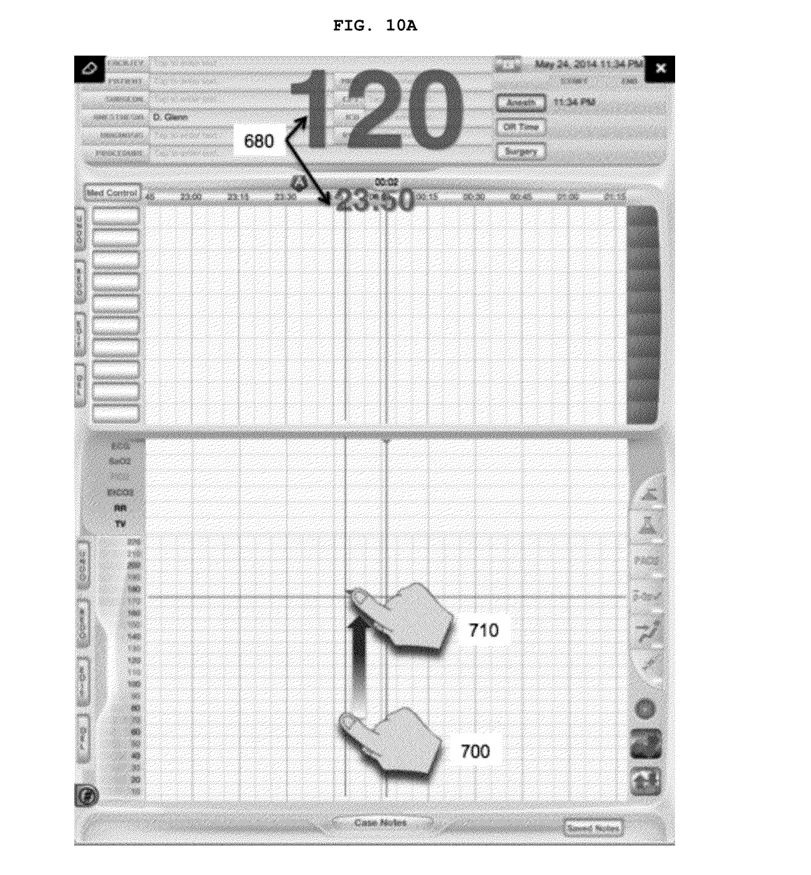

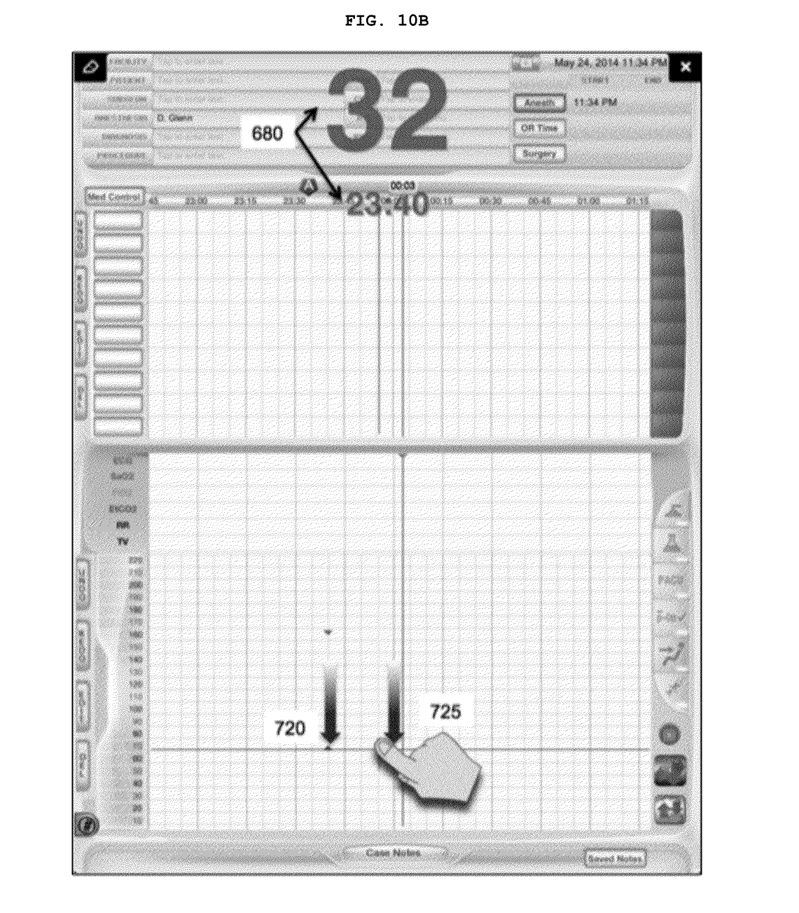

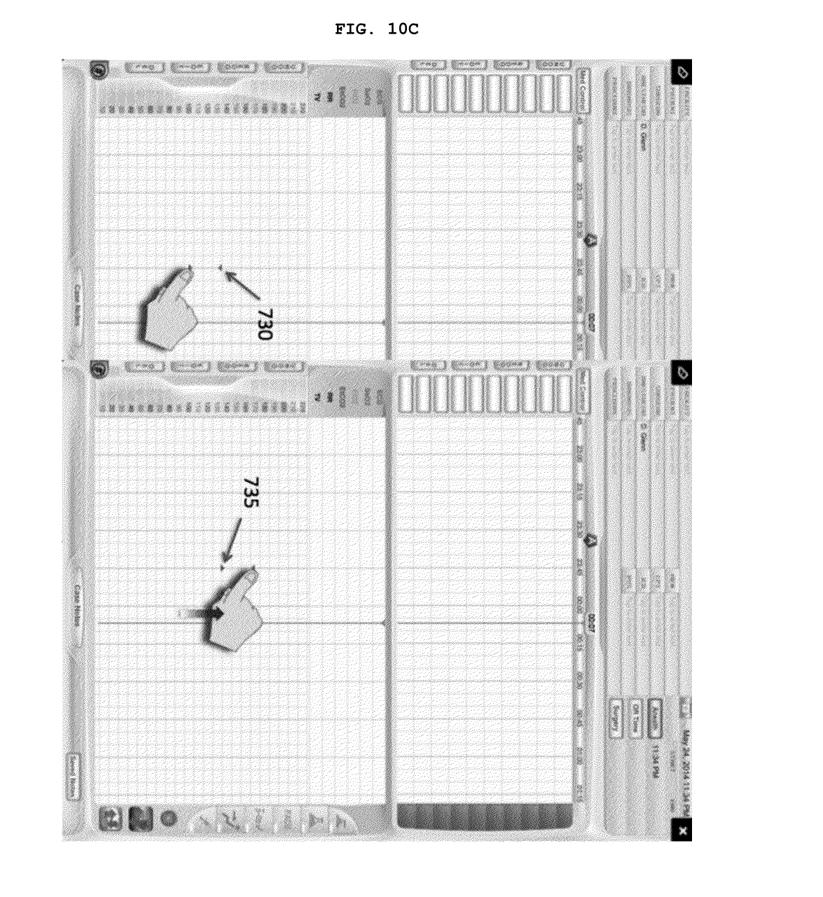

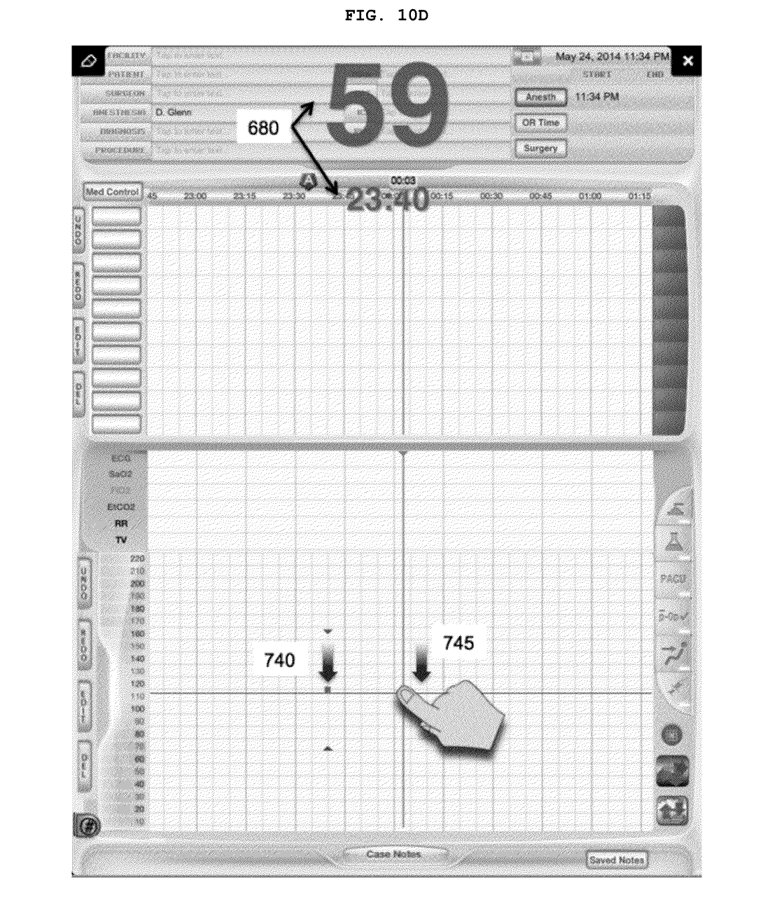

In default mode, the application program enables the anesthesiologist to enter one blood pressure value at a time (FIG. 10A-FIG. 10D) via gesture-based means. Generally, the application program operates in a snap-to-grid mode and requires values be placed exactly on a vertical grid line.

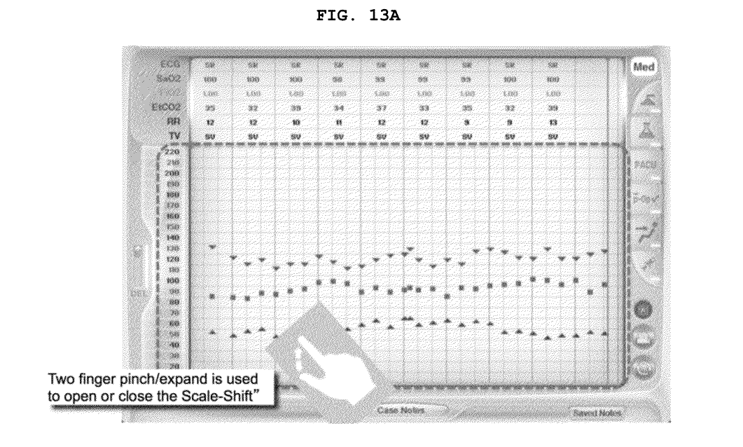

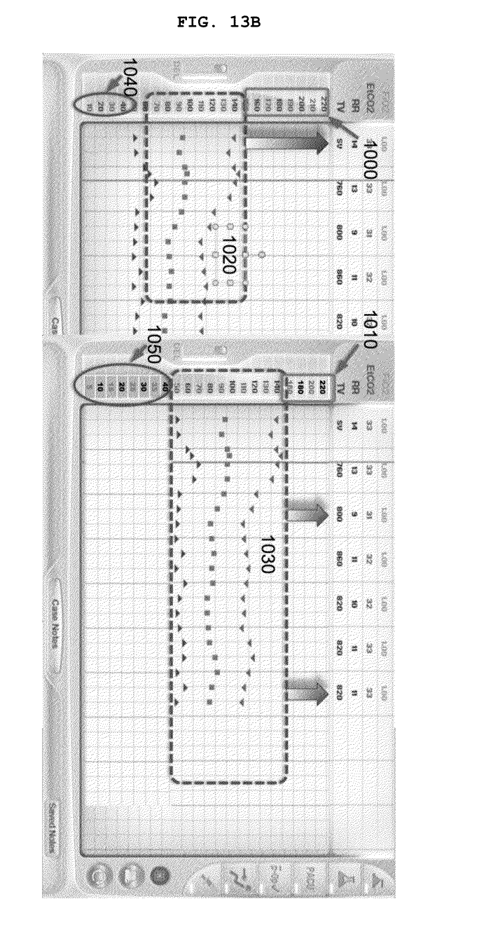

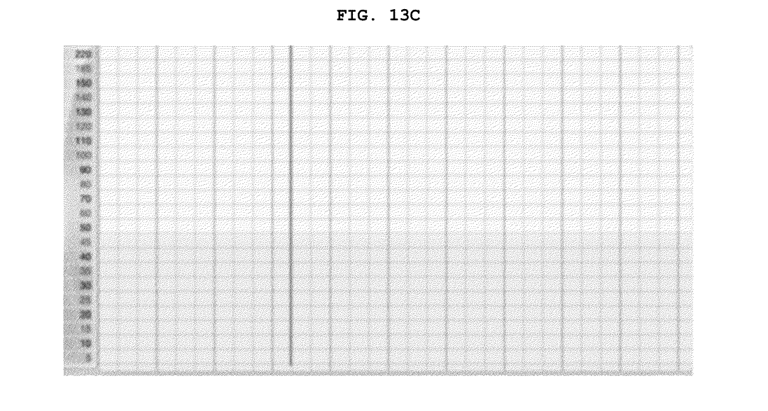

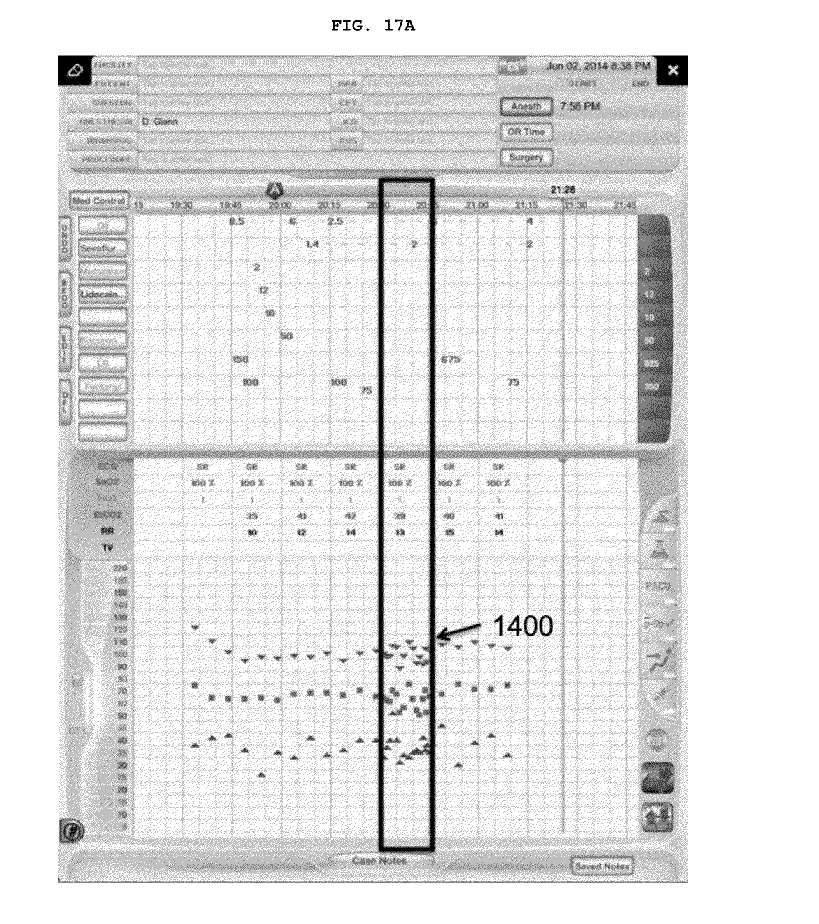

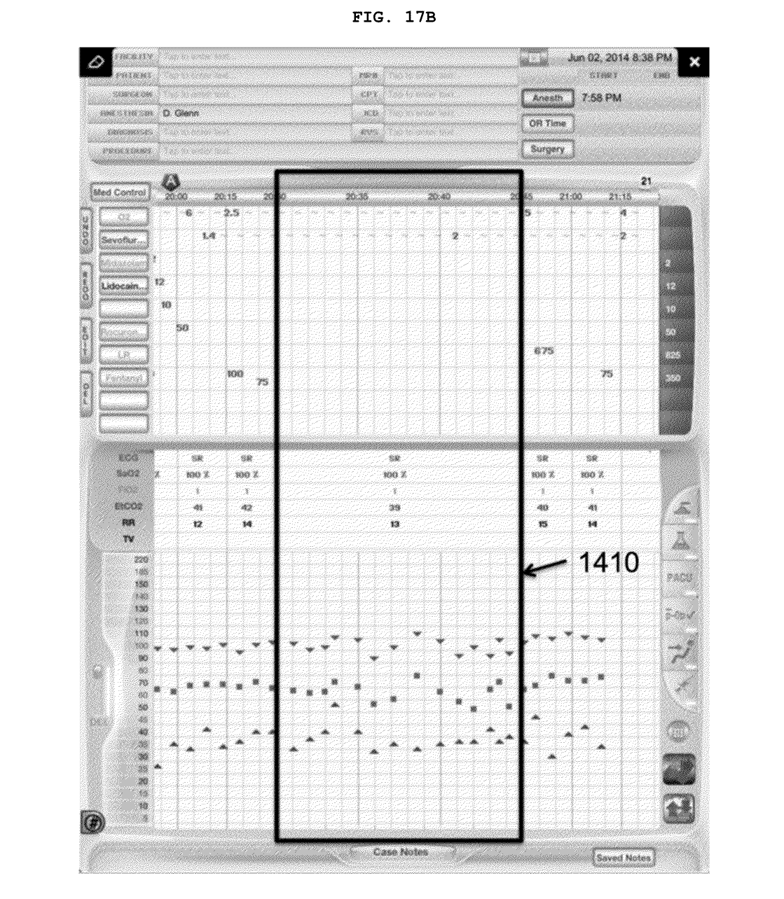

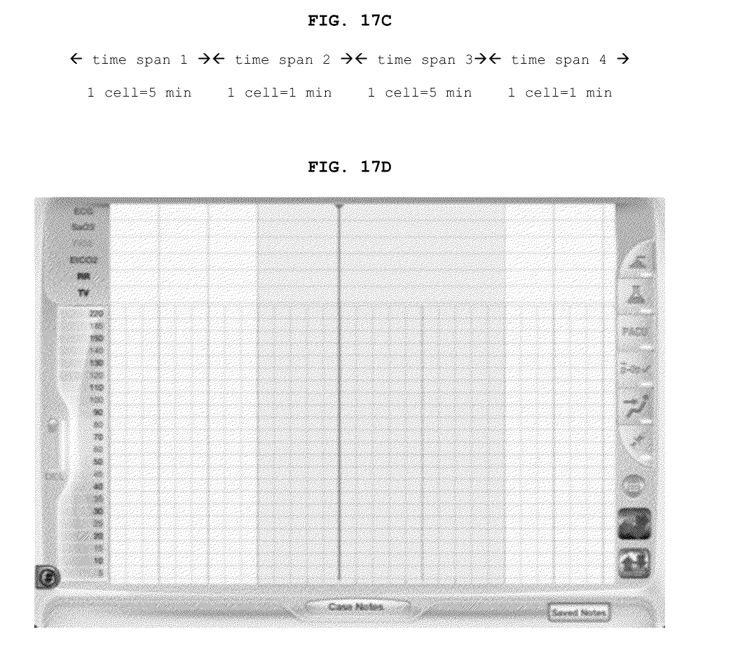

The anesthesiologist can scale-shift within the second sub-view via a gesture (two finger pinch or expand) (FIG. 13A-FIG. 13C). Scale-shifting allows the anesthesiologist to increase the mmHg resolution of the rows in the lower mmHg range. Some blood pressures have only a small difference between the systolic and diastolic values making it difficult for the anesthesiologist to discern the values visually. Increasing the mmHg range between rows accentuates the differences visually. Further, the anesthesiologist can time-shift within the second sub-view over a multitude of non-overlapping time intervals. Time-shifting allows the anesthesiologist to increate the column resolution from one column=5 minutes to one column=1 minute (FIG. 17A-FIG. 17G).

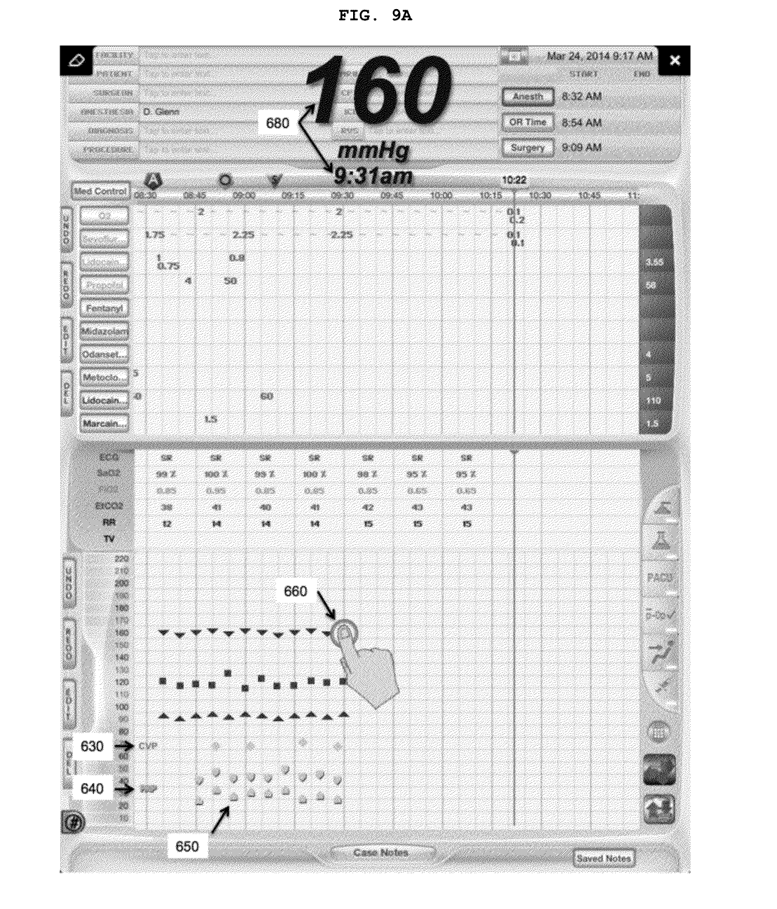

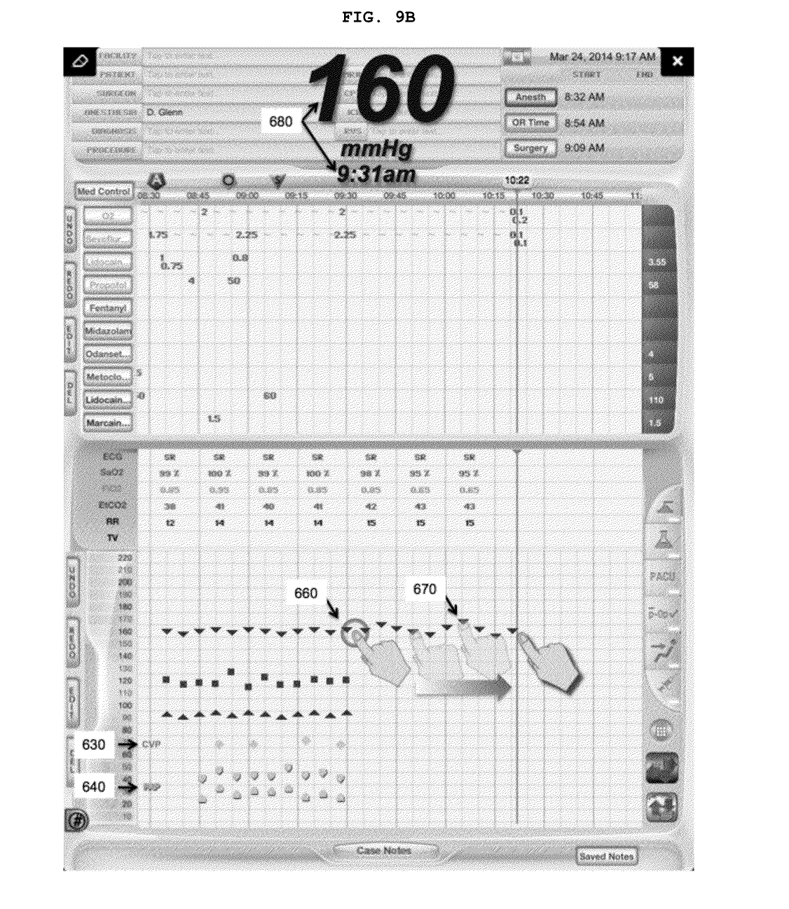

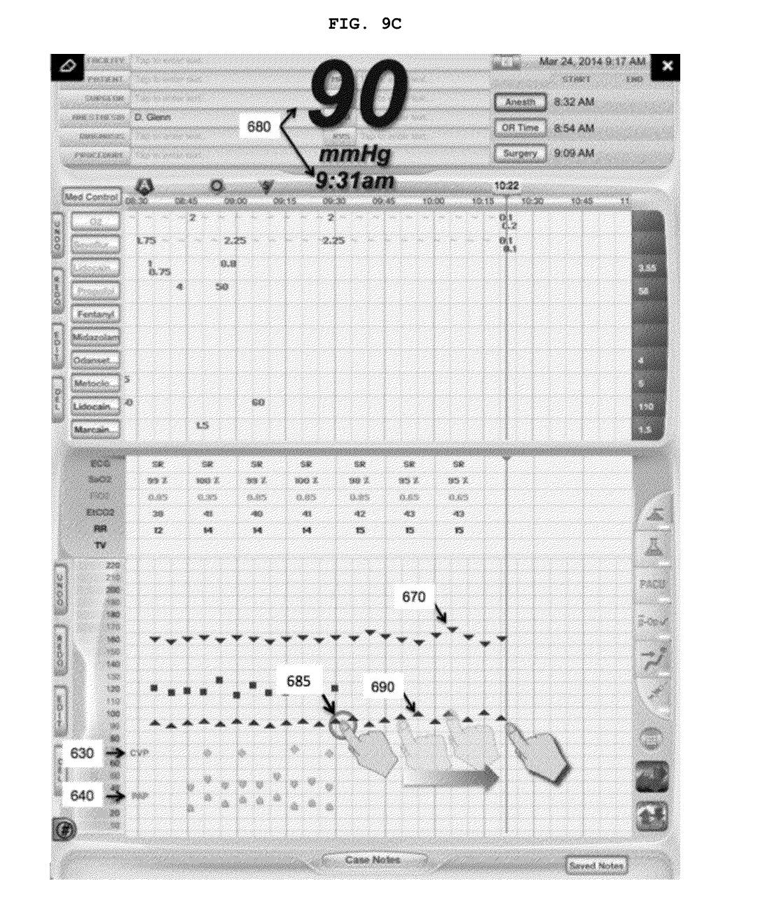

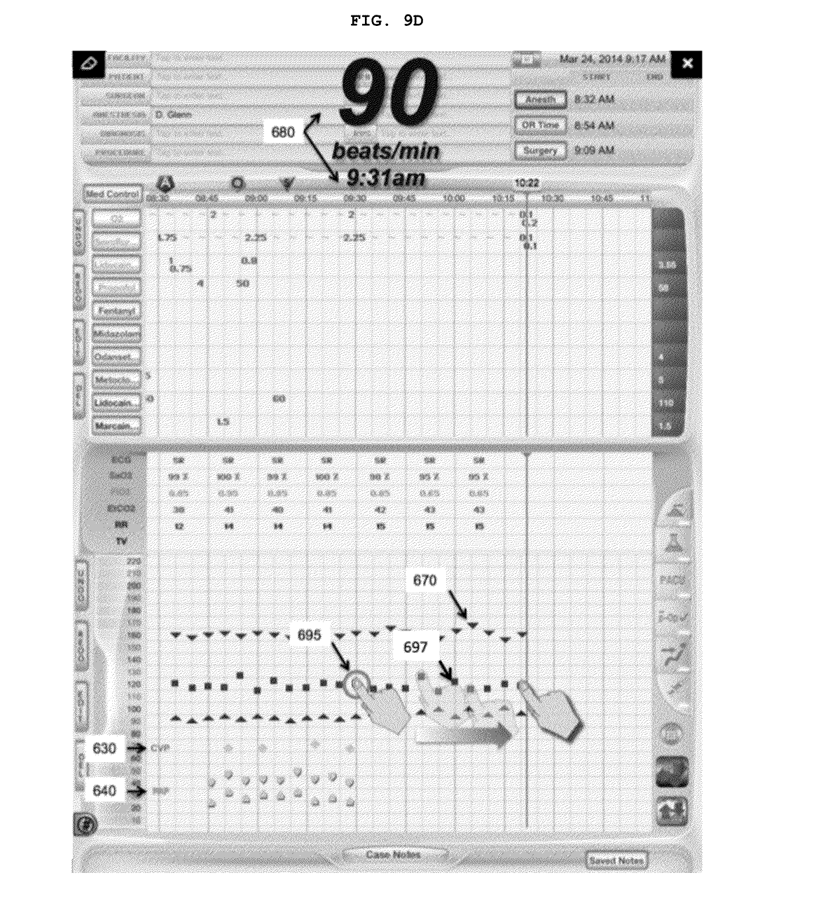

In mass-add mode, the application program enables the anesthesiologist to enter a plurality of blood pressure values (FIG. 9A-FIG. 9D) via gesture-based means.

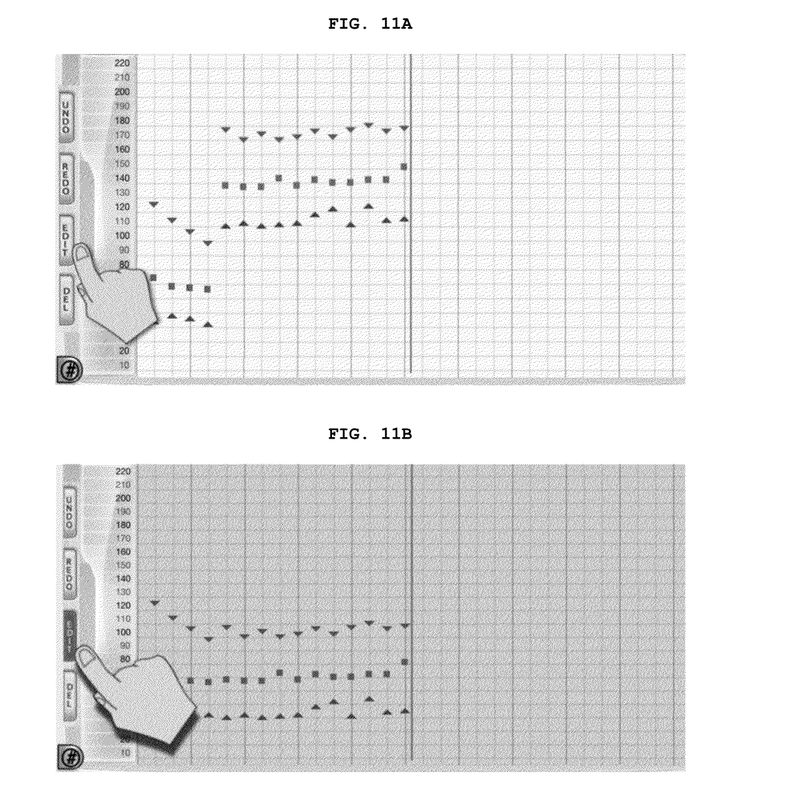

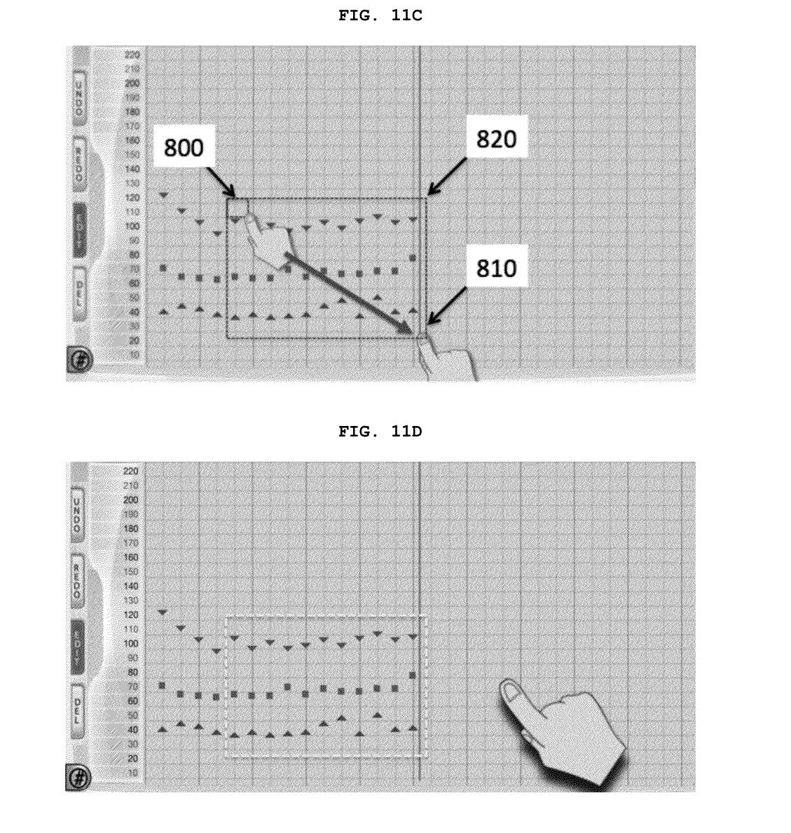

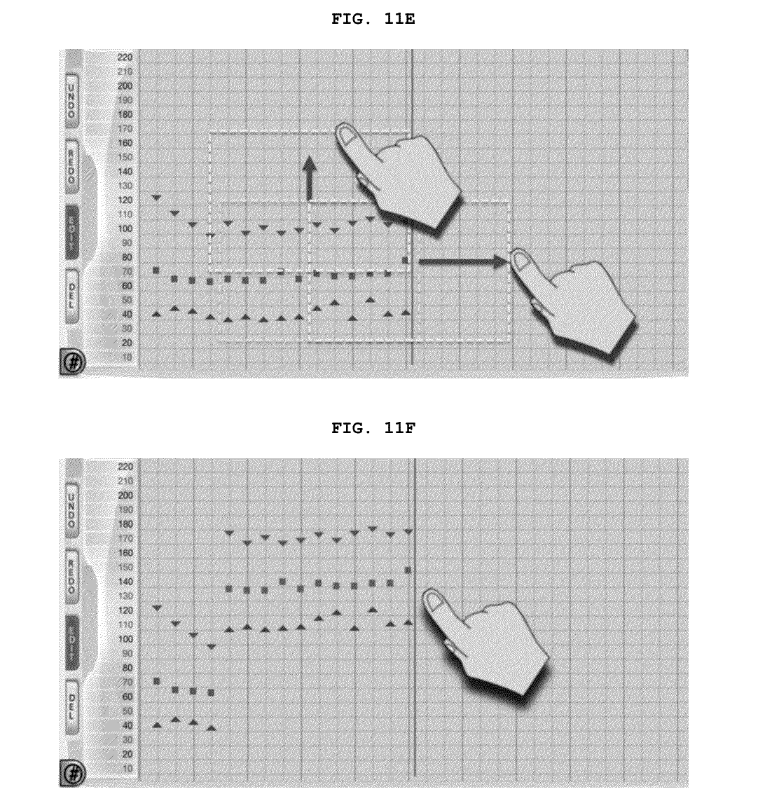

In edit mode, the application program enables the anesthesiologist to change the location within the second sub-view of a plurality of blood pressure values (mmHg/time) (FIG. 11A-FIG. 11F) via gesture-based means.

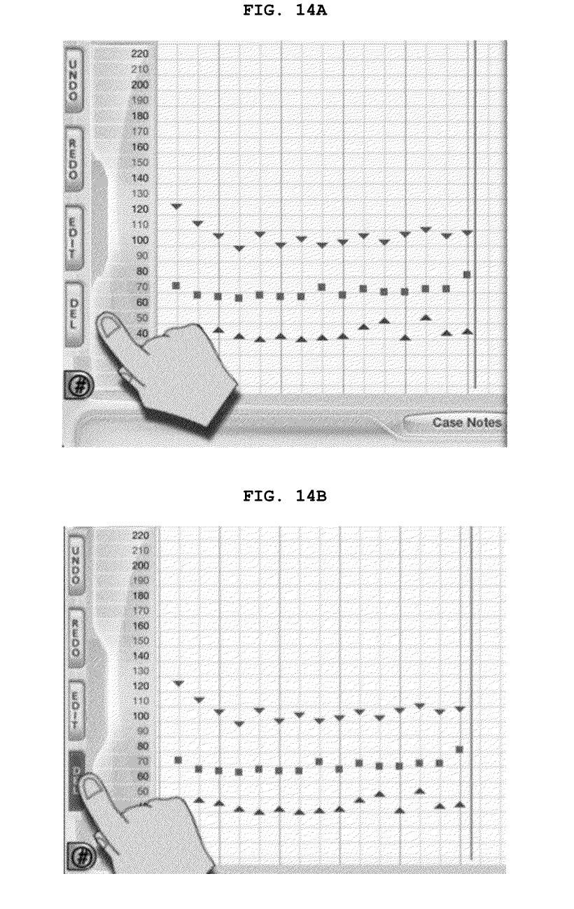

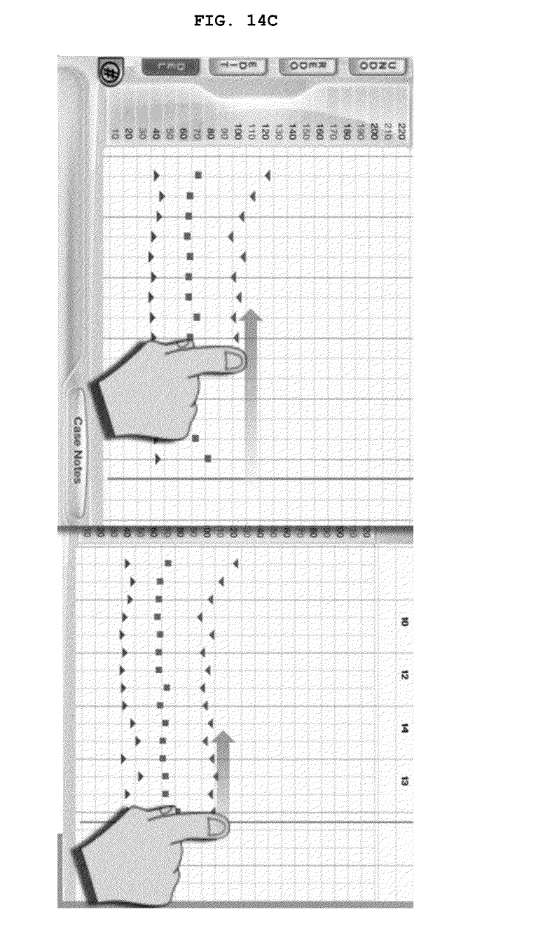

In delete mode, the application program enables the anesthesiologist to remove blood pressure values (FIG. 14A-FIG. 14C) via gesture-based means.

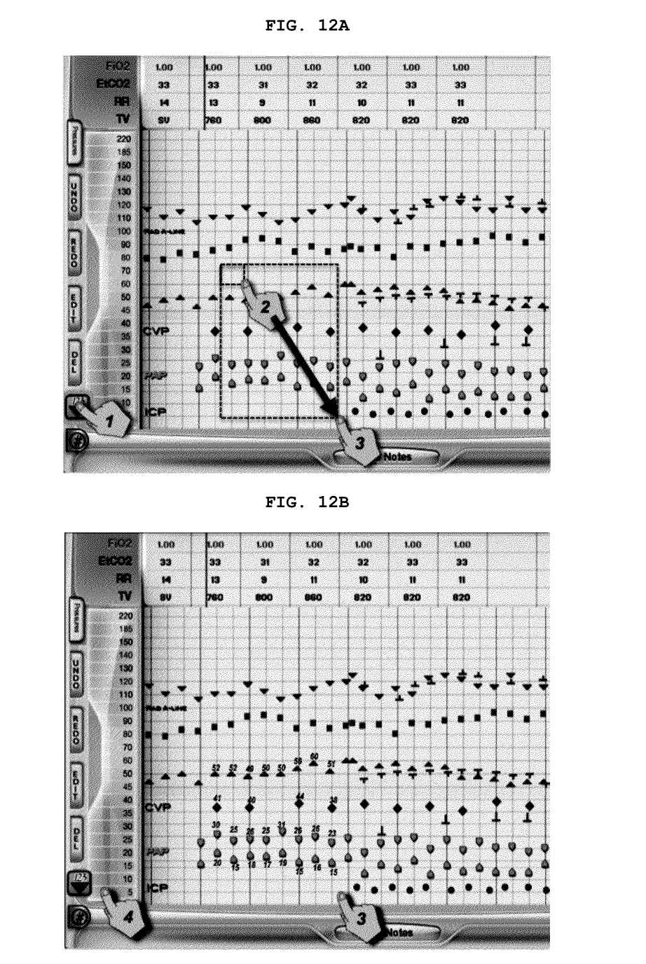

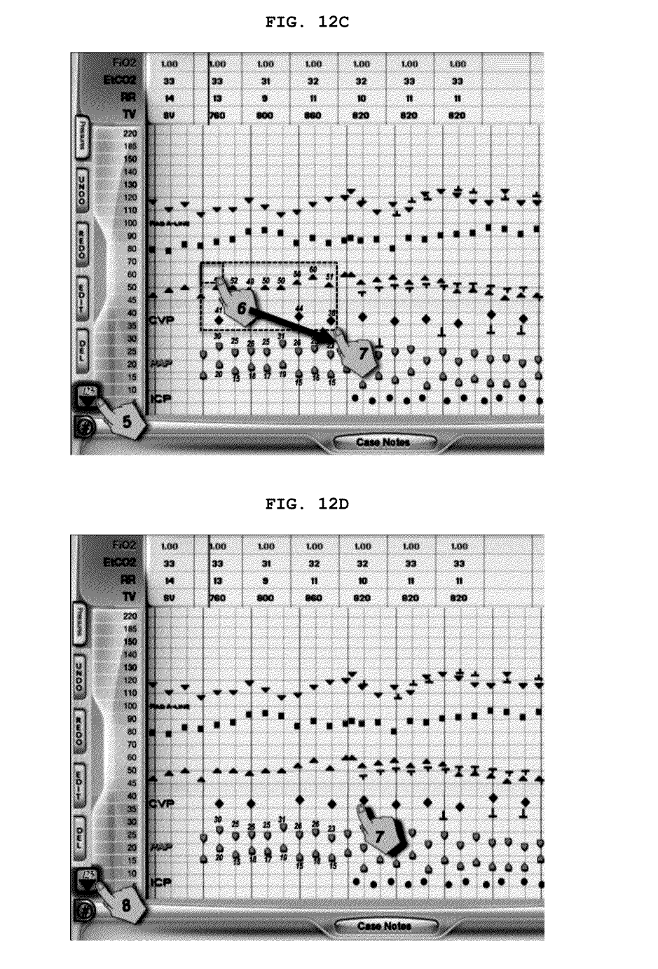

In inspect mode, the application program enables the anesthesiologist to examine the pressure (mmHg) values within a four-sided bounded area of the second sub-view (FIG. 12A-FIG. 12D) via gesture-based means. The second sub-view can become visually “busy” because the application program is tracking several types of blood pressures simultaneously. The anesthesiologist may need to see the pressure (mmHg) value for a group of values since the graphical widget representing the pressure value does not show the mmHg value by default.

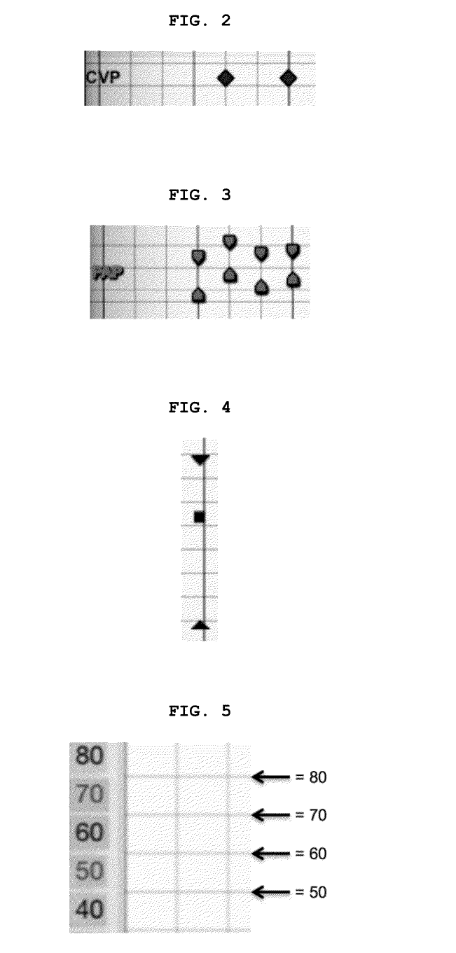

As used in this invention, the definition of “blood pressure vital sign content” is the sum of all blood pressures managed by the application program in a given electronic anesthesia record. TABLE 1, COLUMN A provides a list of many of the blood pressures. The type of surgery determines which blood pressures the anesthesiologist will monitor. Blood pressures come in three types (TABLE 1, COLUMN B) and typically, a given blood pressure, from case to case, will have the same type, however, that is not a rule of medicine. For example, the anesthesiologist may not be able to get a reliable systolic pressure so the mean pressure is used (type 1), when typically in most patients, systolic and diastolic are available (type 2). Additionally, there are times when a surgery necessitates, that the anesthesiologist hook up a transducer and record the pressure. This is an example of a custom blood pressure. The three types of blood pressure managed by the invention, they are:
Type 1. This type tracks the mean of the systolic and diastolic pressures. These values have one part per unit of time (FIG. 2). Instrumentation collecting the blood pressure usually provides the mean and is read by the anesthesiologist and recorded.
Type 2. This type has two parts, the systolic and diastolic pressures (FIG. 3). The anesthesiologist enters systolic first, diastolic second, both aligned to the same time (vertically). If the anesthesiologist enters the diastolic and the value is higher than the systolic, the application program will reverse the entries and take the second value as the systolic and convert the first value to diastolic (FIG. 10C). The application program will require the anesthesiologist to enter both values before continuing to the next recording.
Type 3. This type has three parts, systolic, diastolic and the heart rate (FIG. 4). The anesthesiologist enters systolic first, diastolic second, and heart rate third, all three aligned to the same time (vertically). However, if more than one type 3 is being recorded, the application program will only require the anesthesiologist to record the heart rate once and not for each type 3. There are times when the anesthesiologist is collecting a type 3 value and the surgeon must stop the patient's heart, such as when the patient is put on bypass. In such as case, the application program enables the anesthesiologist to turn off heart rate tracking (not shown).

TABLE 1

| BLOOD PRESSURE NAME (A) | TYPICAL TYPE (B) |
|---|---|
| Central Venous Pressure (CVP) | 1 |
| Intercranial Pressure (ICP) | 1 |
| Mean Arterial Pressure (MAP | 1 |
| Pulmonary Artery Pressure (PAP) | 2 |
| Non-Invasive Blood Pressure (NIBP) | 3 |
| Radial Artery Pressure (RAD-A-Line) | 3 |
| Femoral Pressure (FP) | 3 |
| Carotid Cross Clamp Pressure (CCCP) | 1 or 2 |
| Right Ventricular Pressure (LVP) | 3 |
| Left Ventricular Pressure (LVP) | 3 |
| Brachial Pressure (BP) | 3 |
| Antecubital Pressure (ACP) | 3 |
| Anterior Tibial Pressure (ATP) | 3 |
| Spinal CSF Pressure (CCSFP) | 1 |

The electronic anesthesia record tracks an assortment of times, current time being represented prominently (FIG. 1, 30) in the second sub-view as a vertical line which auto-increments forward (to the right) as time progresses.

The third sub-view displays a timeline of the electronic anesthesia record based on time of day (FIG. 1, 25) with a plurality of time increments vertically aligned to the columns of the second sub-view. To change the time under view in the second sub-view, the anesthesiologist can horizontally scroll right (back in time) (FIG. 19A) or horizontally scroll left (forward in time) (FIG. 19B). It is important to note, that the timeline displayed by the application program in the third sub-view is time of day and not elapsed time. The third sub-view also provides a means of visually tracking time-based events related to the electronic anesthesia record as icons, such as, but not limited to the anesthesia start time (FIG. 1, 26), operating room entry time (FIG. 1, 27) and surgical incision time (FIG. 1, 28). A more complete, non-comprehensive list of time-based events the application program is capable of tracking is listed in TABLE 2. There are two primary reasons the application program tracks time-based events.
The time an anesthesiologist spends in a particular event influences his/her compensation. The general rule being, more time, more compensation.
For regulatory reasons, the occurrence of particular events must be tracked to ensure that they occur and that they occur in a particular order.

TABLE 2

| EVENT NAME | DESCRIPTION |
|---|---|
| Anesthesia Start Time | Time the anesthesiologist assumes |
| | responsibility for the care of the |
| | patient |
| Operating Room Entry | The time the patient physically enters |
| Time | the operating room |
| Anesthesia Turn-Over | The time after the anesthesiologist |
| Time | has induced anesthesia such that the |
| | surgery can proceed. This is when the |
| | surgical nurses can now start prepping |
| | the surgical site on the patient |
| Turnover to Surgeon | Time when surgical nurses finish |
| Time | prepping and surgeons can begin. |
| Surgical Incision Time | The time the surgeon makes an invasive |
| | maneuver or intervention, i.e. scalpel |
| | incision to the skin, injection of |
| | medication in the surgical site |
| Surgery Finish Time | The time when the surgeon is finished |
| | with any invasive maneuver or |
| | intervention but before he/she, the |
| | surgical assistant or nurse begins |
| | applying the surgical wound dressings |
| | or casts, etc. |
| Surgical Finish Time | The time the surgeon, surgical |
| | assistants, or nurses finishes |
| | applying the surgical wound dressings |
| | or casts, etc. |
| Anesthesia Emergence | The time when the anesthesiologist |
| Time | begins to wake up the patient this |
| | typically but not always starts at the |
| | end of the surgical finish time, |
| | sometimes there is overlap |
| OR End Time | The time the patient physically leaves |
| | the operating room |
| PACU Start Time | The time the patient enters the post- |
| | anesthesia care unit, (recovery room) |
| Anesthesia End Time | The time the anesthesiologist finishes |
| | the anesthesia care of the patient by |
| | transferring the responsibility of the |
| | care of the patient over to the |
| | recovery (PACU) nurses |

The fourth sub-view will display information under gesture near the top of the application program window (FIG. 1, 10) above all other user interface components (FIG. 9B, 680). When the anesthesiologist is performing a gesture such as a touch and hold or single finger slide in the second sub-view, it is difficult to know what value, along with its corresponding units (mmHg/time), is under gesture simply because the exact grid position is not visible to the anesthesiologist through his/her finger. In addition, a finger is not a precise instrument so it would be difficult for the anesthesiologist to “sense” the value under gesture. The data shown in the fourth sub-view allows the anesthesiologist to focus on performing the gesture while easily seeing its corresponding value with units. As the gesture progresses, the information displayed in the fourth sub-view updates in real-time. When the gesture ends, the application program hides the fourth sub-view. The application program displays the fourth sub-view near the top of the application program window because it obstructs the least important data the anesthesiologist needs during a surgery, which is the patient name, medical coding info, etc. Once a gesture begins, and the anesthesiologist is managing real-time blood pressure vital sign content, this information is not relevant. Less useful choices of placement include near the focus of the gesture and it would “float” as the gesture progresses. This has the potential of blocking needed information.

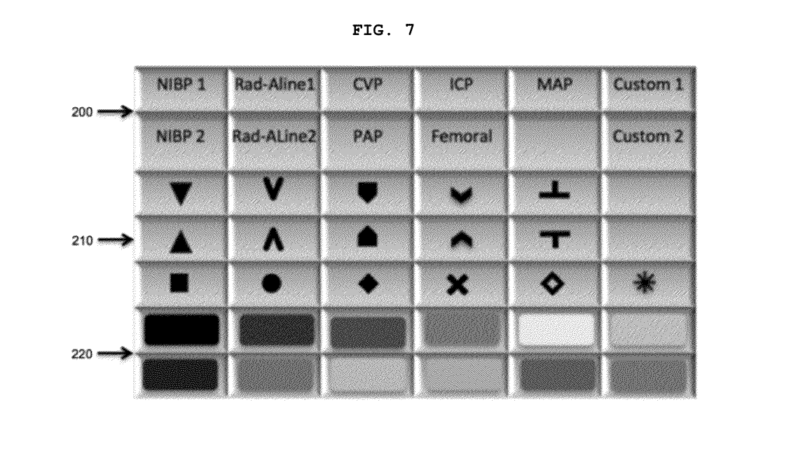

The fifth sub-view contains an apparatus to add new blood pressure types to track in the second sub-view. The anesthesiologist can add a blood pressure type via a gesture (single-finger tap) over the “Pressure” user interface control (not shown) causing the application program to display the fifth sub-view (FIG. 7). The fifth sub-view contains a palette of choices comprising a first group; a second group and a third group such that only one choice can be made from the first and third group. The first group is comprised of plurality of buttons; each enabled to toggle between selected and non-selected states and representing blood pressure types (FIG. 7, 200). The second group is comprised of a plurality of buttons; each enabled to toggle between selected and non-selected states and representing a geometric shape to be associated to the blood pressure type (FIG. 7, 210). The third group is comprised of a plurality of buttons, each enabled to toggle between selected and non-selected states and representing the color of the selected geometric shape from the second group (FIG. 7, 220). The anesthesiologist selects geometric figures in combinations based on the blood pressure value type. Type 1 blood pressure values require one geometric figure; Type 2 requires two; Type 3 requires three. If the anesthesiologist selects a type 1 blood pressure value from the first group, then the application program will require a single selection from the second group of buttons. Similarly, type 2 will require two selections and type 3, three.

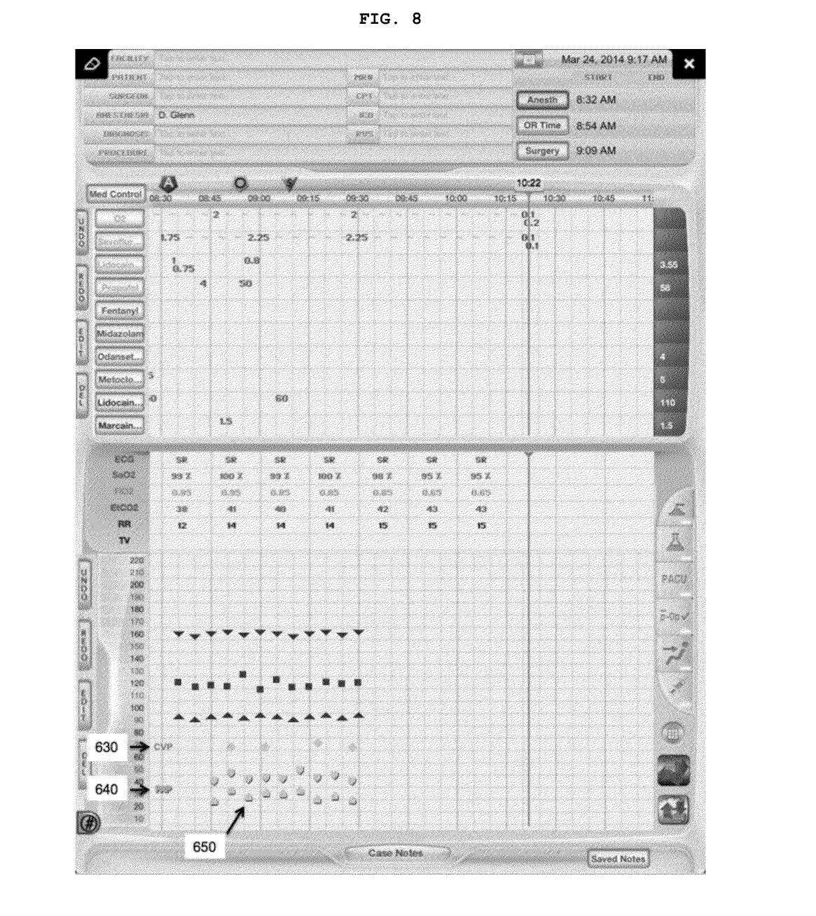

The application program will operatively enable one user interface control per blood pressure to switch the focus between blood pressures under management (FIG. 8, 630, 640). The user interface controls only appear after the anesthesiologist adds the second blood pressure to the electronic anesthesia record. The application program will only allow the anesthesiologist to enter values for one blood pressure at a time. The application program enables the anesthesiologist to move the user interface control to any location in the second sub-view. The initial placement of the user interface control depends on the blood pressure type. For example, ICP has very low mmHg values, so the application program places the selector to the far left of the second sub-view at about 7 mmHg. Once a selector is gestured (single finger tap), the application program will allow its corresponding blood pressure type to accept new values and edit existing values, exclusive of other displayed blood pressure types. Once selected, values associated with other blood pressure types as seen in the second sub-view, fade and become monochrome in color to enable the anesthesiologist to focus visually on the selected blood pressure (FIG. 8, 650).

In another embodiment of the invention, the application program operatively enables a process to allow the anesthesiologist, via gesture based means, to enter blood pressure or heart rate values in mass on a multi-function gesture-sensitive device. FIG. 9A-FIG. 9D represents a scenario where current time has advanced beyond where the anesthesiologist has entered values. This may happen when the anesthesiologist must turn his/her attention away from the application program and to the patient. At a time medically feasible, the anesthesiologist will enter the values observed to catch up to current time. In this representative example, type 3 value will be input. The anesthesiologist will perform the following process for entering, in mass, a type 3 blood pressure and heart rate value:
The anesthesiologist performs a first gesture (single-finger touch and hold) on the second sub-view over the systolic part of a pre-existing blood pressure value (FIG. 9A, 660).
The application program will respond to the first gesture in the second sub-view by displaying the blood pressure and time values corresponding to the systolic part of a pre-existing type 3 value in the fourth sub-view (FIG. 9A, 680).
While performing a second gesture, different from the first, (single-finger slide to the right), the application program will input systolic values in the configured shape (FIG. 7, 210) and in the configured color (FIG. 7, 220) with one value per grid column (placed exactly on the grid column line) until the second gesture ends and not to go beyond current time (FIG. 1, 30) (FIG. 9B, 670). Some of the values might be slightly inaccurate from actual values at a given time. Generally, anesthesiologists consider this acceptable (and preferable) because the data entry shows close enough approximations and trending to actual values. The application program will display the blood pressure value in mmHg and time in real time in the fourth sub-view as the anesthesiologist performs the second gesture.
The anesthesiologist will perform the first gesture again on the second sub-view over the diastolic part of a pre-existing type 3 value (FIG. 9C, 685). Again, the application program will respond to the first gesture in the second sub-view by displaying the blood pressure and time values corresponding to the diastolic part of a pre-existing type 3 value in the fourth sub-view (FIG. 9C, 680).
The anesthesiologist will perform the second gesture again and the application program will input the diastolic part of the type 3 value until the second gesture ends (FIG. 9C, 690). Again, the application program will display the blood pressure value in mmHg and time in real time in the fourth sub-view as the anesthesiologist performs the second gesture.
The anesthesiologist will perform the first gesture again on the second sub-view over the heart rate part of a pre-existing type 3 value (FIG. 9D, 695). Again, the application program will respond to the first gesture in the second sub-view by displaying the heart rate and time values corresponding to the diastolic part of a pre-existing type 3 value in the fourth sub-view (FIG. 9D, 680).
The anesthesiologist will perform the second gesture again and the application program will input the heart rate part of the type 3 value until the second gesture ends (FIG. 9D, 697). Again, the application program will display the heart rate value in beats per minute and time in real time in the fourth sub-view as the anesthesiologist performs the second gesture (FIG. 9D, 680).

Likewise, if the anesthesiologist is attempting to enter a type 1 blood pressure, then the application program will only require step 1-2 be performed (systolic is replaced by the mean blood pressure). If the anesthesiologist is attempting to enter a type 2 blood pressure value, then the application program will only require steps 1-4 be performed.

In another embodiment of the invention, the application program operatively enables a process to allow the anesthesiologist, via gesture-based means, to enter blood pressure and heart rate values individually on a multi-function gesture-sensitive device. The application program will not allow the next blood pressure value to be entered until the anesthesiologist has entered all required parts of the current blood pressure value and in a particular order, the application program controlling the sequencing. In this representative example, a type 3 value will be input. The anesthesiologist will perform the following process for entering a type 3 value:
The anesthesiologist performs a first gesture (single-finger touch and hold) to specify location of the systolic part on the second sub-view at or near the desired pressure and time (FIG. 10A, 700).
The application program will respond to the first gesture by displaying a crosshair, the center of which being the grid location under gesture and will display the pressure and time of the systolic part corresponding to the center in the fourth sub-view (FIG. 10A, 680).
Relying on the data displayed in the fourth sub-view, the anesthesiologist will optionally perform a second gesture (single-finger slide) to adjust the exact position of the crosshair center and hence the systolic pressure and time as necessary (FIG. 10A, 710).
If snap-to-grid is active, then the application program will place values only on vertical grid lines, else they can go between grid lines.
The anesthesiologist will again perform the first gesture to specify the location of the diastolic part on the second sub-view at or near the desired pressure. The application program will align diastolic part to the time of the systolic part. In this case, the application program does not display the crosshair, however, it shows only a horizontal bar. If the anesthesiologist specifies a pressure for the diastolic greater than the systolic pressure, in mmHg, then the application program swaps the interpretation of the parts. The application program will reinterpret the diastolic part as the systolic part and vice-versa and will swap their respective geometric shapes (FIG. 10C, 730, 735).
Relying on the data displayed in the fourth sub-view, the anesthesiologist will again optionally perform a second gesture (single-finger slide) to adjust the exact position of the horizontal line and hence the diastolic pressure as necessary (FIG. 10B, 725).
The anesthesiologist will again perform the first gesture to specify the location of the heart rate part on the second sub-view at or near the desired heartbeats per minute value. The application program will align heartbeat part to the time of the systolic part. In this case, as with the diastolic part, the application program does not display the crosshair, rather, it shows only a horizontal bar (FIG. 10D, 740).
Relying on the data displayed in the fourth sub-view, the anesthesiologist again will optionally perform a second gesture (single-finger slide) to adjust the exact position of the horizontal line and hence the heart rate as necessary (FIG. 10D, 745).

If the anesthesiologist is attempting to enter a type 1 blood pressure, then the application program will only require step 1 be performed (systolic is replaced by the mean blood pressure). If the anesthesiologist is attempting to enter a type 2 blood pressure value, then the application program will require steps 1-2 be performed.

In another embodiment of the invention, the application program operatively enables a process to allow the anesthesiologist, via gesture-based means, to edit existing blood pressure vital sign content on a multi-function gesture-sensitive device. Edit means to move a blood pressure value to a different pressure/heart rate and/or time. Occasionally, an anesthesiologist may enter a large number of blood pressure values at a wrong value. It rarely happens that just one value is in error since the anesthesiologist would easily notice it. Rather, it is more common for a whole set to be off and assumed correct. To edit an existing value(s), the process is as follows:
The anesthesiologist will perform a first gesture (single-finger tap) that will toggle the second sub-view into an edit mode (FIG. 11A). The application program, to facilitate a better user experience, will change the background color of the second sub-view, alerting the anesthesiologist of the mode change (FIG. 11B).
The anesthesiologist performs a second gesture (FIG. 11C, 800) (single-finger touch and hold), different from the first, the focus of which forms an initial vertex of a rectangle.
The anesthesiologist performs a third gesture different from the first and second (single-finger slide) and moving away from the initial vertex (FIG. 11C, 810). The application program tracks the progress of the third gesture by displaying a rectangle outlined as dotted lines starting at the initial vertex and whose farthest vertex is the current focus of the third gesture (FIG. 11C, 820). Upon completion of the gesture, the rectangle remains (FIG. 11D).
The anesthesiologist performs a fourth gesture (single-finger touch and slide) over any part of the rectangle. The application program will respond to the fourth gesture by moving the rectangle to track the focus of the fourth gesture (FIG. 11E). Upon completion of the fourth gesture, the application program will relocate all blood pressure vital sign content bounded by the rectangle from their original position in the blood pressure vital sign grid to their new position as determined by the location of the bounding rectangle and their relative location therein (FIG. 11F).
The anesthesiologist will perform a first gesture a second time, which will toggle the second sub-view back to default mode. The application program will change the background color of the second sub-view back to its original color, alerting the anesthesiologist that the second sub-view is no longer in edit-mode (FIG. 11A).

In another embodiment of the invention, The application program operatively enables a process to allow the anesthesiologist to inspect or de-inspect specific blood pressure or heart rate values on a multi-function gesture-sensitive device. As used in this invention, “inspect” shall mean to display additional information about at least one blood pressure value. By default, the application program displays only a geometric figure at some time and mmHg/heart rate value. The actual mmHg/heart rate value is not shown to help reduce clutter and to help the anesthesiologist focus on the overall trend of the data being tracked. However, occasionally the anesthesiologist must see the actual numeric values. To show the exact blood pressure and heart rate values, the anesthesiologist will perform the following process:
The anesthesiologist performs a first gesture (FIG. 12A, 1) (single-finger tap on the “Show Numericals” user interface control). The application program puts the second sub-view into inspect mode where it is ready to respond to the second gesture.
The anesthesiologist performs a second gesture (FIG. 12A, 2) (single-finger touch and hold), different from the first, the beginning of which forms the initial vertex of a rectangle.
The anesthesiologist performs a third gesture different from the first and second (single-finger slide) away from the initial vertex. The application program tracks the progress of the third gesture by displaying a rectangle outlined as four dotted lines starting at the initial vertex and whose farthest vertex is the current focus of the gesture. All blood pressure and heart rate values bounded by the area of the rectangle will have their respective values displayed near the geometric figure representing the value (FIG. 12B).
The process ends when the anesthesiologist performs the first gesture for a second time (finger taps over the “Show Numericals” user interface control) (FIG. 12D, 4). The application program returns the second sub-view to default mode.

In another embodiment of the invention, the application program operatively enables a process to allow the anesthesiologist, via gesture-based means, to grid-shift or change the resolution of the blood pressure and heart rate scale on a multi-function gesture-sensitive device. This embodiment enables the anesthesiologist to closely examine blood pressure values whose differences are very fine and yet very relevant. For example, the anesthesiologist must monitor blood pressures taken in the brain or lungs with a high degree of resolution because the difference between their systolic and diastolic values is small, which is not the case in arterial pressures. The application program divides the second sub-view into three horizontal regions. The first region is from 0-49 mmHg, and the second is from 50-150 mmHg and the third is from 151-220 mmHg. By default, the horizontal rows in the all three regions increment by 10 mmHg. The anesthesiologist may require a resolution change in the first region from 10 mmHg to 5 mmHg or even 1 mmHg per row. To grid-shift the blood pressure scale, the anesthesiologist will perform the following process:
The anesthesiologist will perform a first gesture (single-hand, two-finger vertical expand) (FIG. 13A) that will increase the resolution between the blood pressure values in the first horizontal region. The shift occurs by increasing the resolution in the region from 0-49 (FIG. 13B, 1040) from one row=10 mmHg to one row=5 mmHg (FIG. 13B, 1050). The region from 50 mmHg to 150 mmHg is unchanged but shifted up (FIG. 13B, 1020 and FIG. 13B, 1030). The region from 151 mmHg to 220 mmHg is compressed and shifted from 1 row=10 mmHg (FIG. 13B, 1000) to one row=20 mmHg (FIG. 13B, 1010). The application program will change the background color of the first horizontal region of the second sub-view for easy identification (FIG. 13C).
In this optional step, the anesthesiologist can further increase the resolution between the blood pressure values by repeating the first gesture. The shift occurs by increasing the resolution in the region from 0-49 (FIG. 13B, 1050) from one row=5 mmHg to one row=1 mmHg (not shown). The application program adjusts the second horizontal region to span 50 mmHg to 220 mmHg while it moves the third horizontal region out of the viewable area of the second sub-view.
The anesthesiologist can reduce the resolution between the blood pressure values by performing a second gesture (two finger pinch). If starting from the end of step 2, then the second gesture will return the blood pressure scale to the end of step 1. If starting from the end of step 1, then the second gesture will return the blood pressure scale to the default resolution of 1 row=10 mmHg and the background of the shifted region will return to its default color.

In another embodiment of the invention, the application program operatively enables a process to allow the anesthesiologist, via gesture-based means, to delete pre-existing blood pressure values on a multi-function gesture-sensitive device. To delete a pre-existing blood pressure value, the anesthesiologist will perform the following process:
The anesthesiologist will perform a first gesture (single finger tap) that will toggle the second sub-view into delete mode (FIG. 13A). The application program, to facilitate a better user experience, will change the background color of the second sub-view, alerting the anesthesiologist of the mode change (FIG. 13B).
The anesthesiologist will perform a second gesture (touch and slide) over a portion of the second sub-view. The application program erases any values passed over by the second gesture (FIG. 13C).
The anesthesiologist will re-perform the first gesture to toggle the second sub-view out of delete mode and into default mode (not shown).

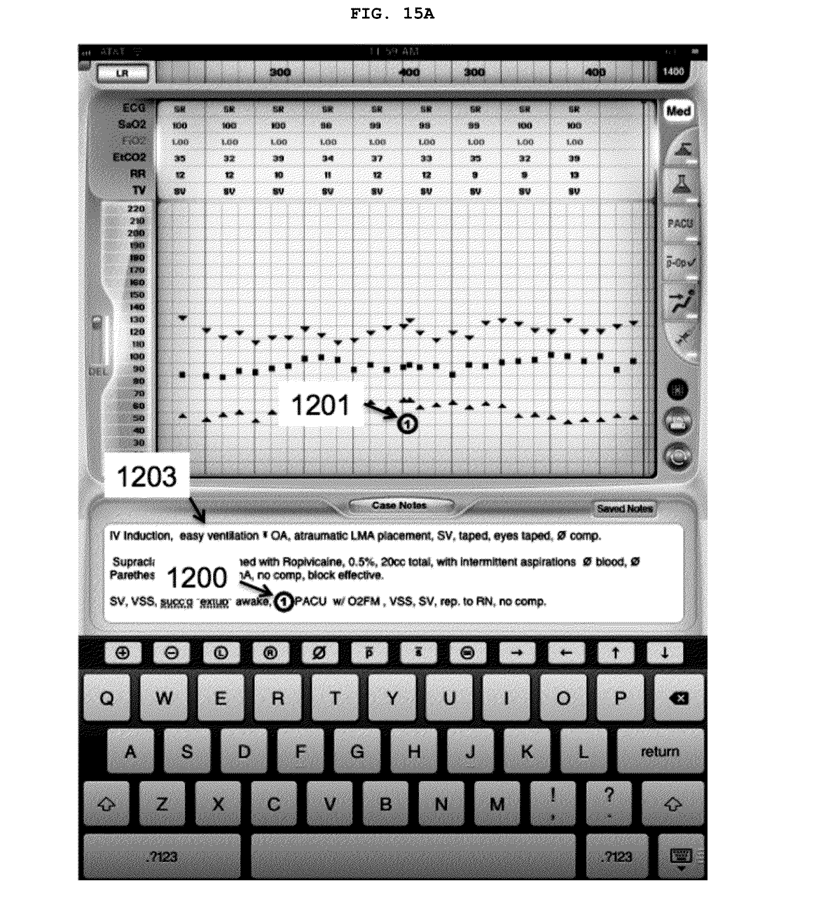

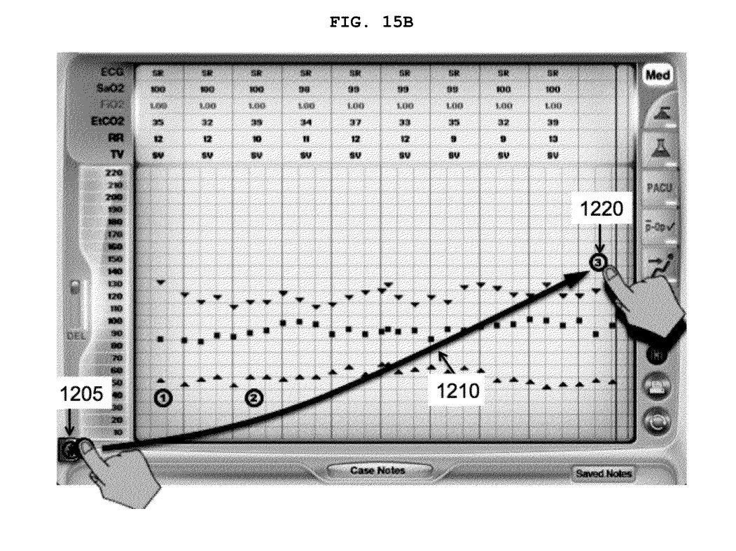

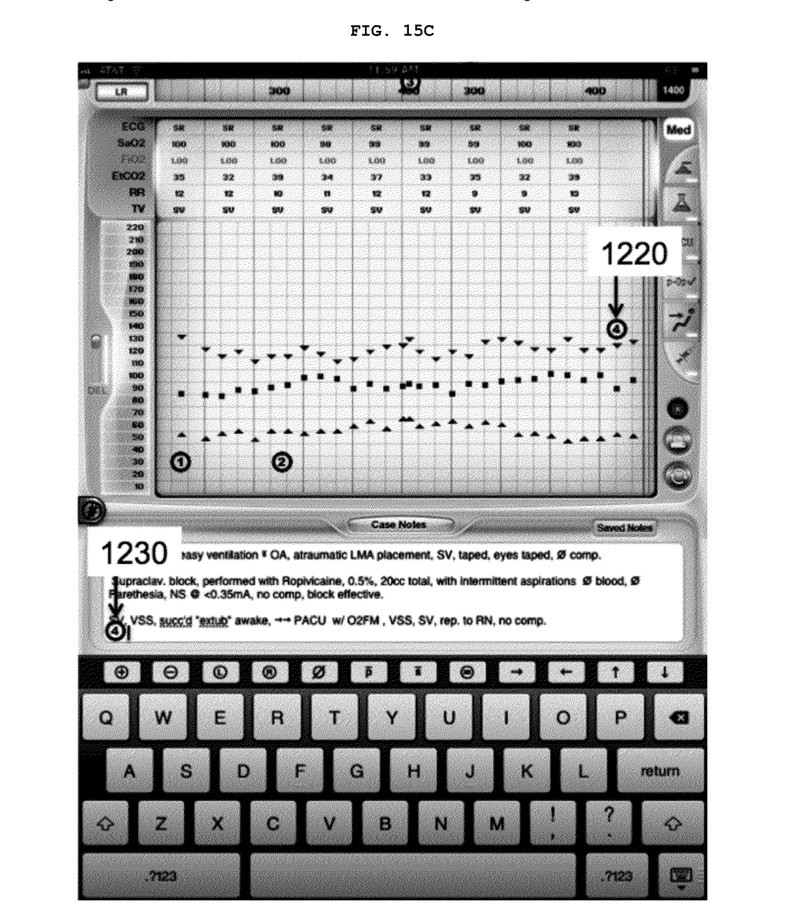

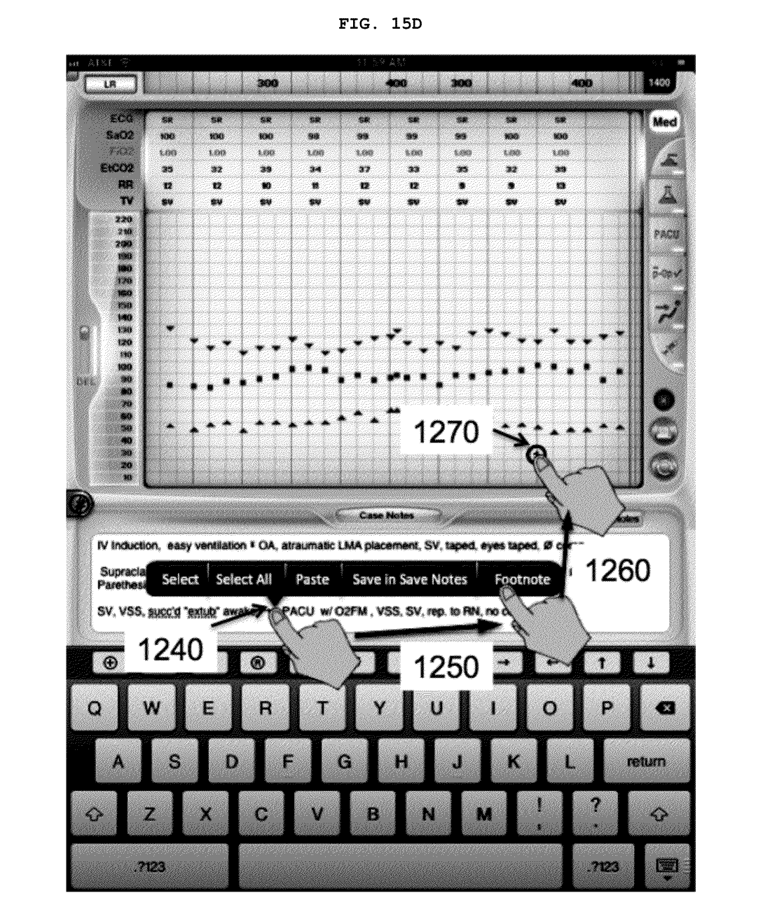

In another embodiment of the invention, the application program operatively enables a process to allow the anesthesiologist, via gesture-based means, to associate case notes to blood pressure vital sign content on a multi-function gesture-sensitive device. The application program enables the anesthesiologist to enter free form text case notes for the electronic anesthesia record as a whole. These case notes can be voluminous and may require a blood pressure and time context to fully realize the meaning of the case note. A case note is comprised of two visual references. The application program represents each visual reference as a circle with a number in the center such that each pair has the same number. The application program will assign a higher number to each newly created visual reference pair. The first visual reference is associated to a pressure and time on the blood pressure vital sign grid (FIG. 15A, 1201). The second visual reference (FIG. 15A, 1200) is associated to the text of the case note. The anesthesiologist can tap on the second visual reference (FIG. 15A, 1200) and the application program will display the blood pressure vital sign grid in the second sub-view at the location the first visual reference. Alternatively, the anesthesiologist can tap on the first visual reference (FIG. 15A, 1201) and the application program will display the case note associated with it, scrolling to it if necessary. To add a new visual reference to a case note, the anesthesiologist will perform the following process:
The anesthesiologist will perform a first gesture (single-finger touch) (FIG. 15B, 1205) leading to the application program displaying the first visual reference as a flashing red circle enclosing the case note identifier at the focus of the gesture. The application program will open the case note editor (FIG. 15A, 1203) and the virtual keyboard. The application program will place the second visual reference at the bottom of the case note editor, below all previous notes. A second visual reference will appear on a new line below any existing case notes. The cursor will appear directly right of the second visual reference (FIG. 15C, 1230).
The anesthesiologist will perform a second gesture (single-finger slide) (FIG. 15B, 1210) to the place the visual reference in the second sub-view (FIG. 15B, 1220) and consequently at a pressure and time in the blood pressure vital sign grid. Upon termination of the second gesture, the application program will change the color of the visual reference to blue.

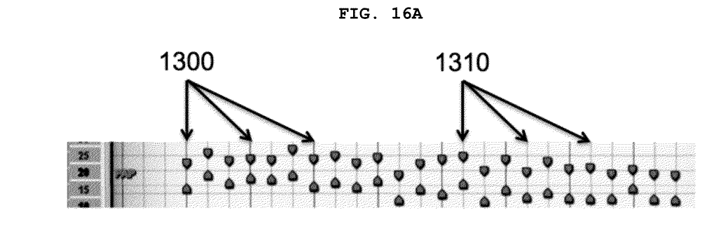

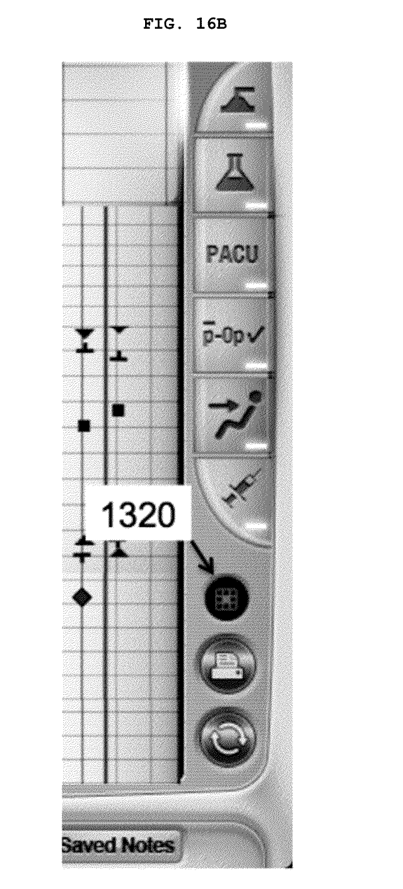

In another embodiment of the invention, the application program operatively enables a process to allow the anesthesiologist, via gesture-based means, to enter blood pressure values guided by a snap-to-grid function on a multi-function gesture-sensitive device. The second sub-view by default displays vertical grid lines in 5-minute increments (FIG. 16A, 1310) with 15-minute increments drawn in a wider pen (FIG. 16A, 1300). The anesthesiologist can scale-shift from a 5-minute increment to a 1-minute increment (FIG. 16B). In such a case, every 5-minute increment is marked in a wider pen. When placing blood pressure values into the second sub-view, the application program will by default place them on a vertical grid line, in effect only allowing values every 1 minute or every 5 minutes depending on the current scale-shift and not between grid lines. However, the anesthesiologist may need to place a blood pressure at a time between increments. For example, if a patient's heart has stopped and doctors are attempting to restart it. To toggle the blood pressure management apparatus into and out of snap-to-grid mode, the process is as follows:
The anesthesiologist will gesture (single finger tap) over a toggle button (FIG. 1, 36 and FIG. 16B, 1320) resulting in the application program turning off the snap-to-grid function. As long as the snap-to-grid function is off, any blood pressure or heart rate values entered will go any time chosen by the anesthesiologist.
The anesthesiologist will gesture (single finger tap) over a toggle button (FIG. 16B, 1320) resulting in the application program turning on the snap-to-grid function. As long as the snap-to-grid function is on, any blood pressure values entered will go a time increment of 1-minute or 5-minute depending on the present scaling of the blood pressure vital sign grid.

In another embodiment of the present invention, the application program will operatively enable a process to allow the anesthesiologist, via gesture-based means, to change the time resolution of the blood pressure vital sign grid, called time-shifting, on a multi-function gesture-sensitive device. There are scenarios that can occur that require blood pressure values be recorded once per minute, or even more frequently, as opposed to the default of once per five minutes. Due to the size of the geometric figures used to record blood pressure values, the second sub-view can appear cluttered and difficult to interpret (FIG. 17A, 1400). Time-shifting allows the anesthesiologist to selectively change the time resolution of the columns in the second sub-view from a lower time resolution to a higher time resolution, in this embodiment, from 1 cell=5 minutes to 1 cell=1 minute (FIG. 17B, 1410). The application program further allows for a plurality of non-overlapping shifted areas of the blood pressure vital sign grid simultaneously (FIG. 17C). To change the time resolution of the blood pressure vital sign grid, the anesthesiologist will perform the following process:
The anesthesiologist will perform a first gesture (single-handed two-finger horizontal expand) (FIG. 17E). The application program determines the region of the second sub-view it will expand by where the anesthesiologist's gesture touches the blood pressure vital sign grid. The first vertical grid lines on the outside of the space between the finger tips, i.e., the grid line immediately to the right of the finger on the right and the grid line immediately to the left of the left finger (FIG. 17F). At the end of the gesture, the region under gesture of the second sub-view will reflect the time of 1 cell=1 minute (FIG. 17G). Upon completion of the gesture, the application program will change the background color of the second sub-view to help visually identify the time-shifted region (FIG. 17D).

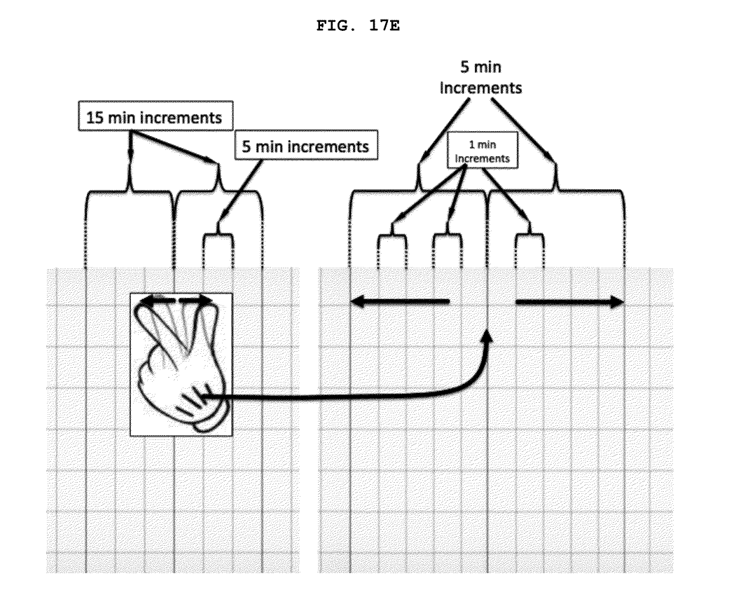

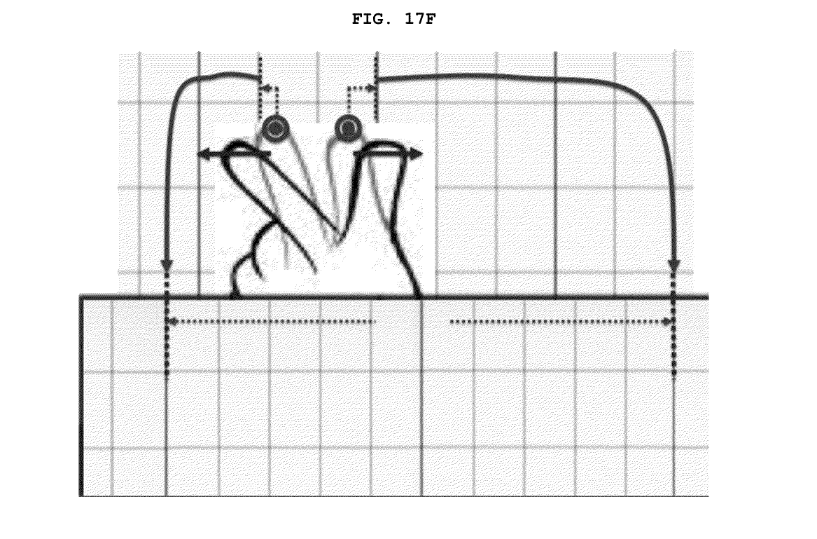

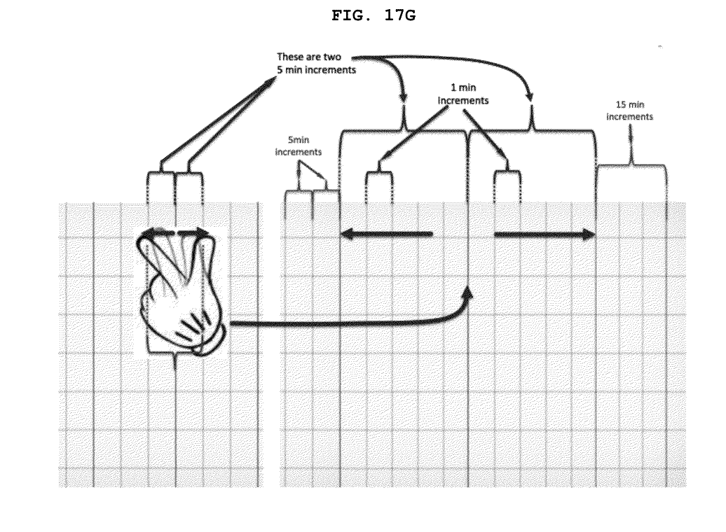

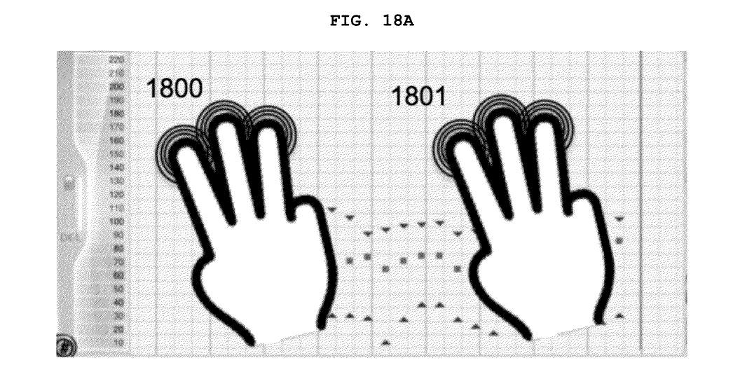

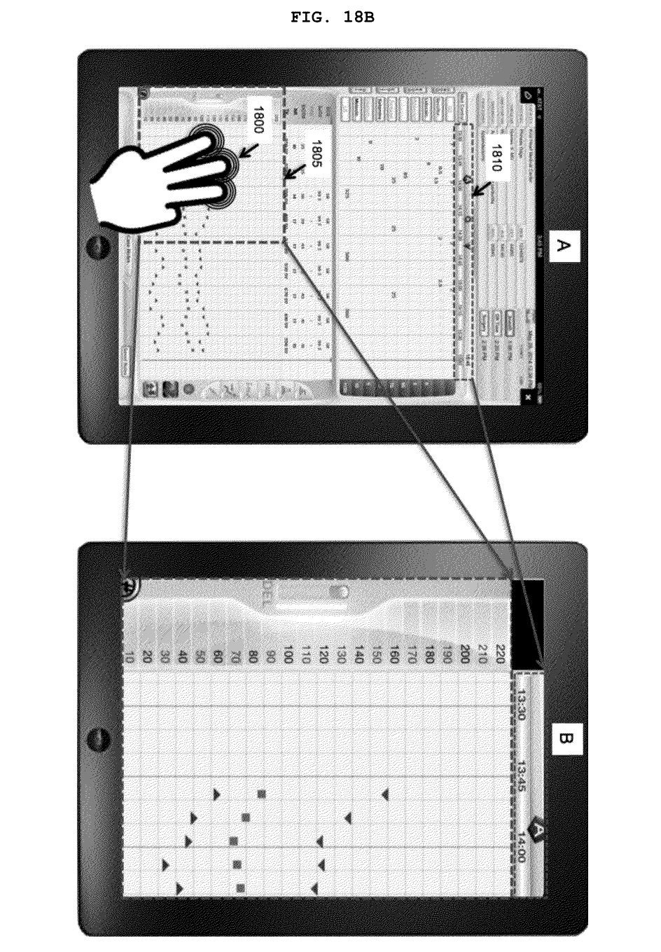

In another embodiment of the invention, the application program operatively enables a process to allow the anesthesiologist, via gesture-based means, to expand and magnify the screen area on a multi-function gesture-sensitive device. This embodiment enables an anesthesiologist to better see application content. The application program divides the combined area occupied by the first and second sub-views into two equal areas. The application program enables each equal area to respond to a gesture (single-hand, triple-finger tap) (FIG. 18A, 1800, 1801). To expand and magnify, the process is as follows:
The anesthesiologist will gesture over one of the two equal areas. In response, the application program will increase the screen area occupied by the first and second sub-views to completely full the application program window (FIG. 1, 10) with the exception of the third sub-view which remains at the top of the application program window (FIG. 18B, 1810). In addition, the application program simultaneously applies a magnification that increases the size of all displayed content by a first magnification of 220%.
The anesthesiologist may optionally perform the gesture a second time anywhere in the application program window and the magnification will increase to a second magnification of 320% (FIG. 18A B).
If the anesthesiologist performed step 2 and then performs the gesture a third time, the application program reduces the magnification back to the first magnification. If after step 3 or if the anesthesiologist did not perform step 2, the application program returns the application program window (FIG. 1, 10) back to the state before the first gesture (FIG. 18A A).

In another embodiment of the invention, the application program operatively enables a process to allow the anesthesiologist, via gesture-based means, to horizontally scroll the blood pressure vital sign grid on a multi-function gesture-sensitive device. The application program enables an anesthesiologist to change the position of the second sub-view relative to the blood pressure vital sign grid. To horizontally scroll the blood pressure vital sign grid to a previous point in time, the anesthesiologist will perform a first gesture (two-finger horizontal slide to the right) (FIG. 19A). To horizontally scroll the blood pressure vital sign grid to a later point in time, the anesthesiologist will perform a second gesture (two-finger horizontal slide to the left) (FIG. 19B).

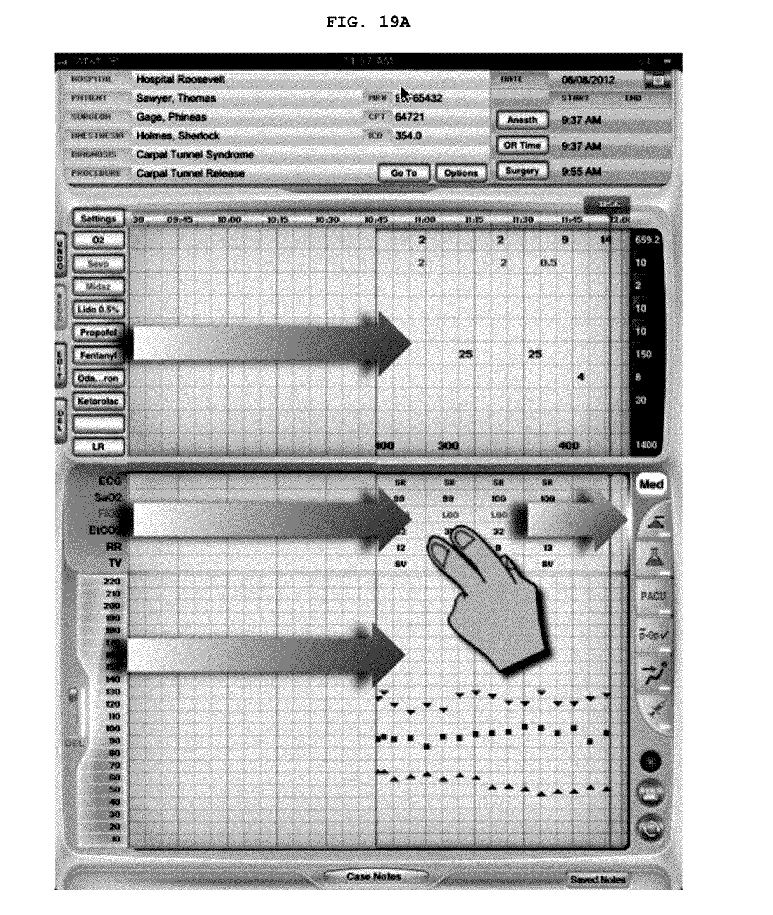

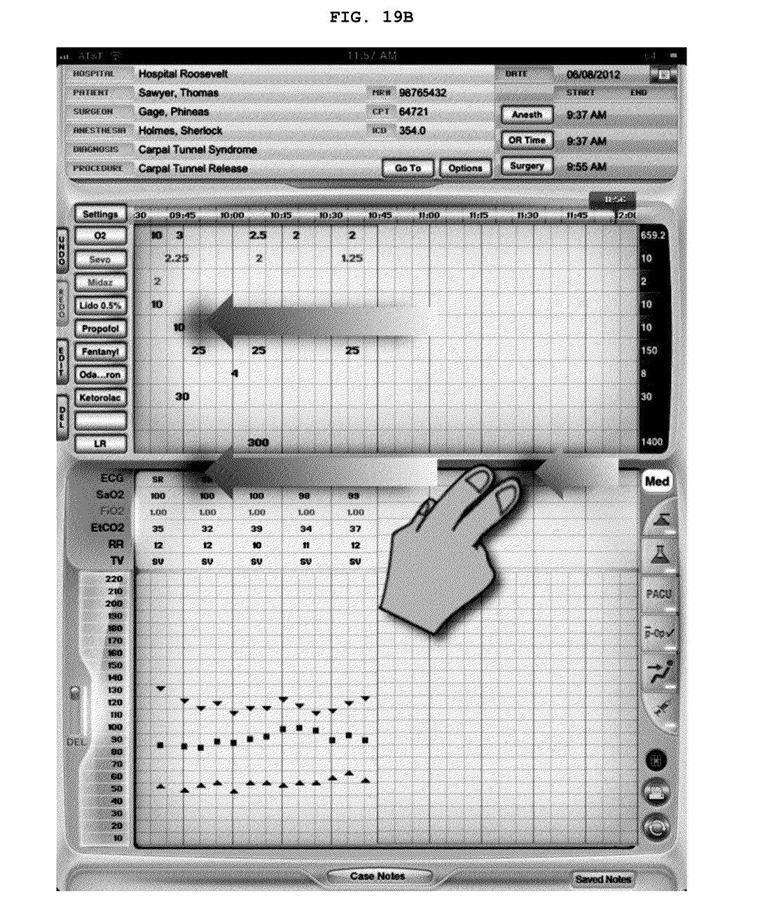

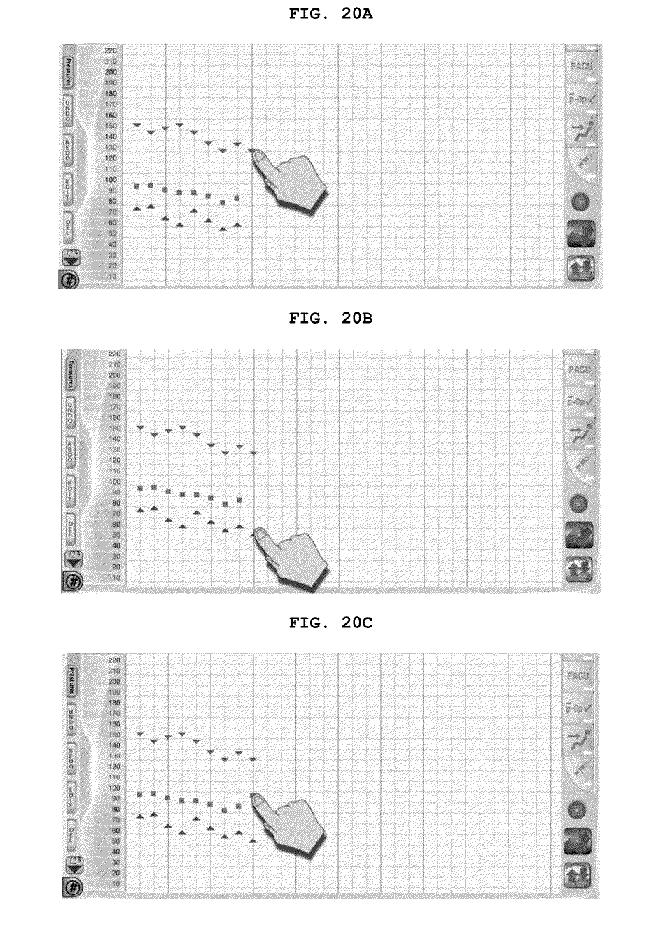

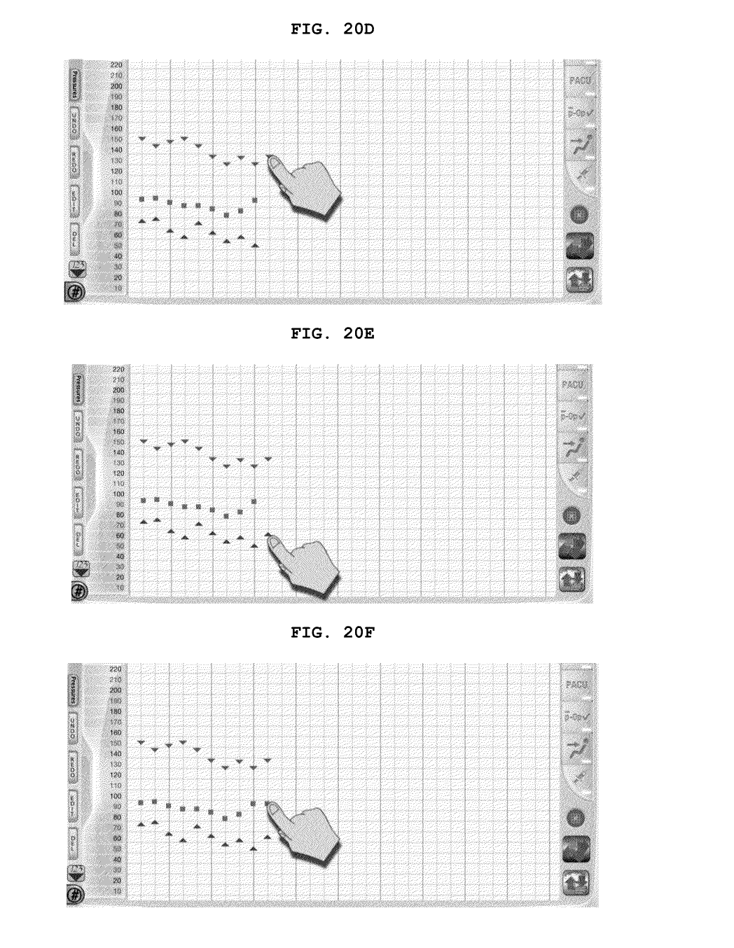

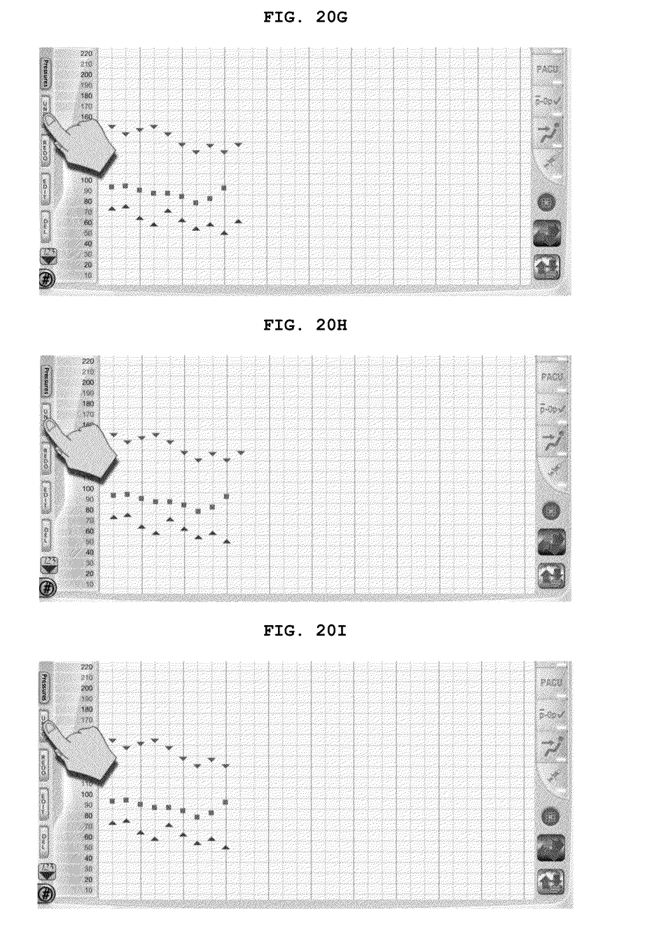

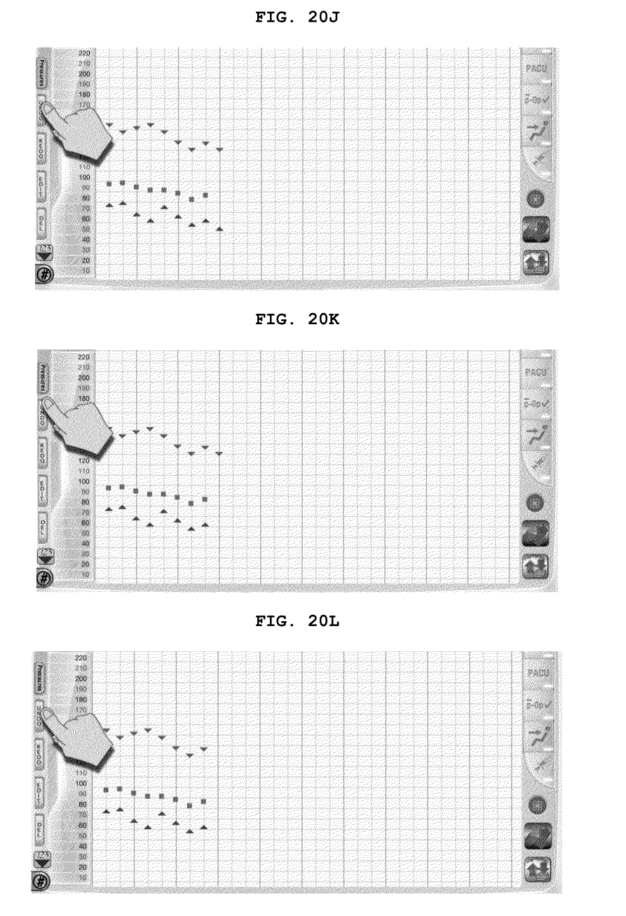

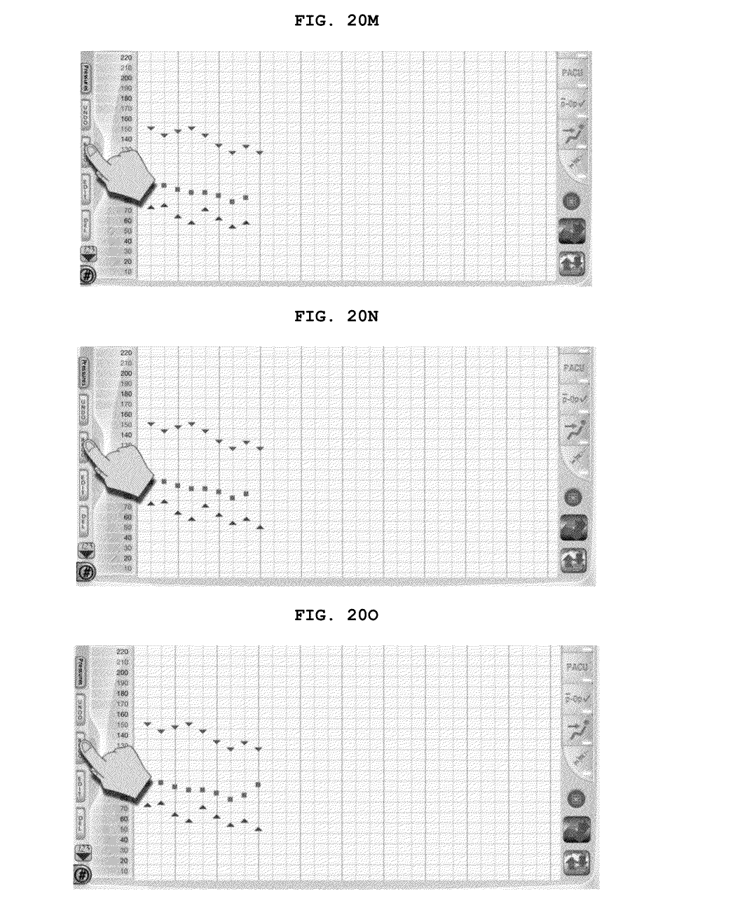

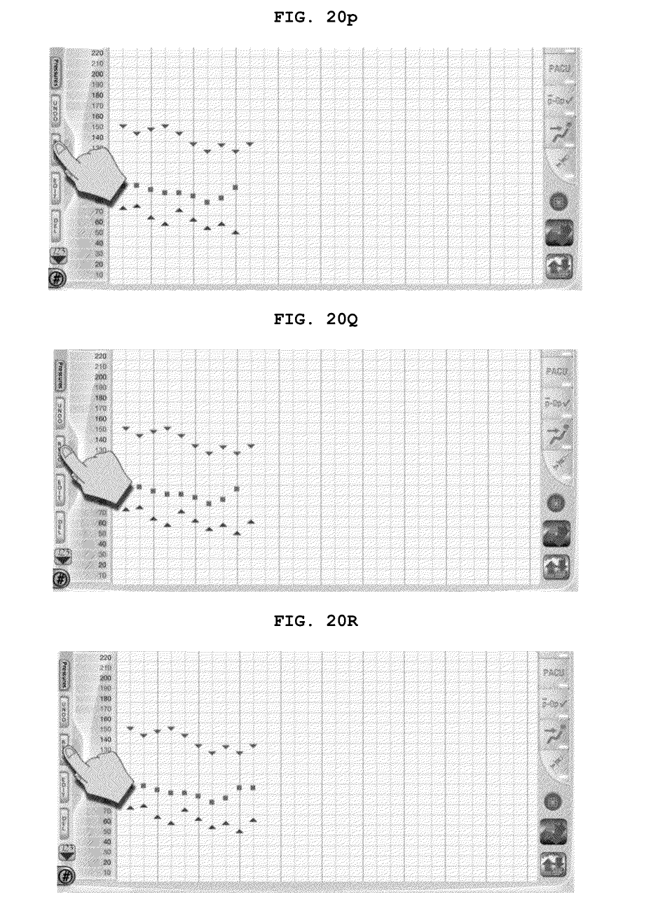

In another embodiment of the invention, the application program operatively enables a means to undo and redo changes to content in the blood pressure vital sign grid on a multi-function gesture-sensitive device. Undo enables an anesthesiologist to undo the previous 1 to N actions performed on the blood pressure vital sign grid. Redo reapplies the next 1 to N previously performed actions but previously undone. In a representative example, the anesthesiologist performs a series of blood pressure vital sign additions (FIG. 20A-FIG. 20F). Then the anesthesiologist performs the undo embodiment six times (FIG. 20G-FIG. 20L), followed by the redo embodiment six times (FIG. 20M-FIG. 20R).

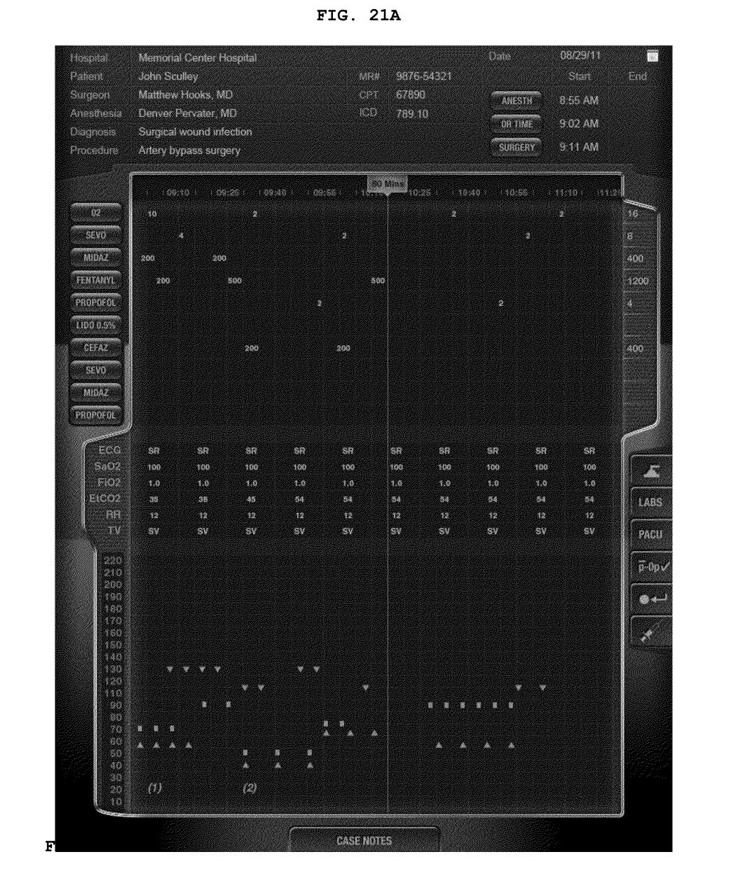

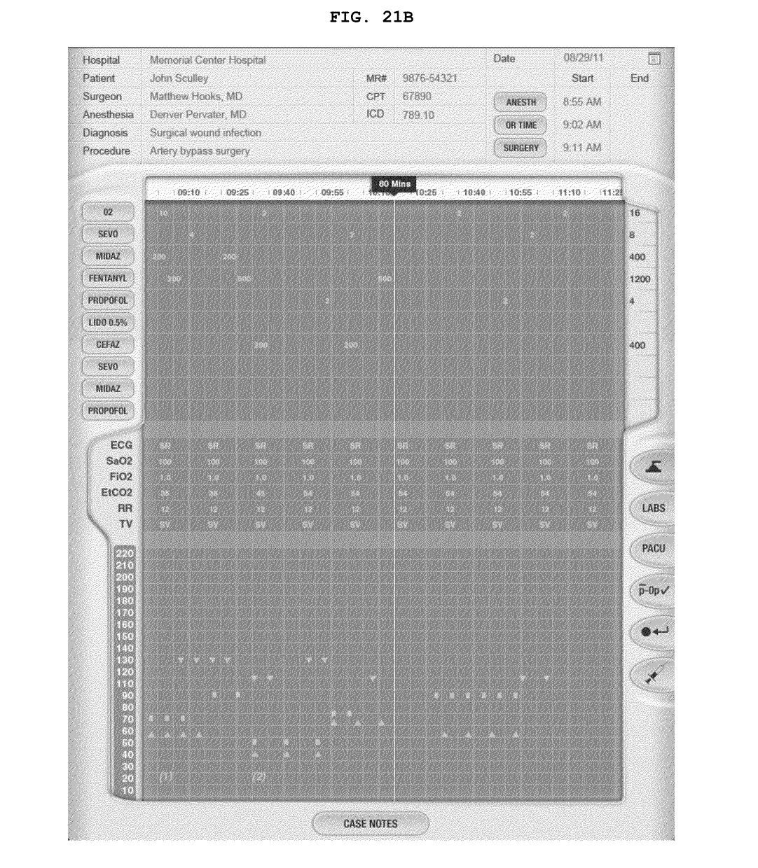

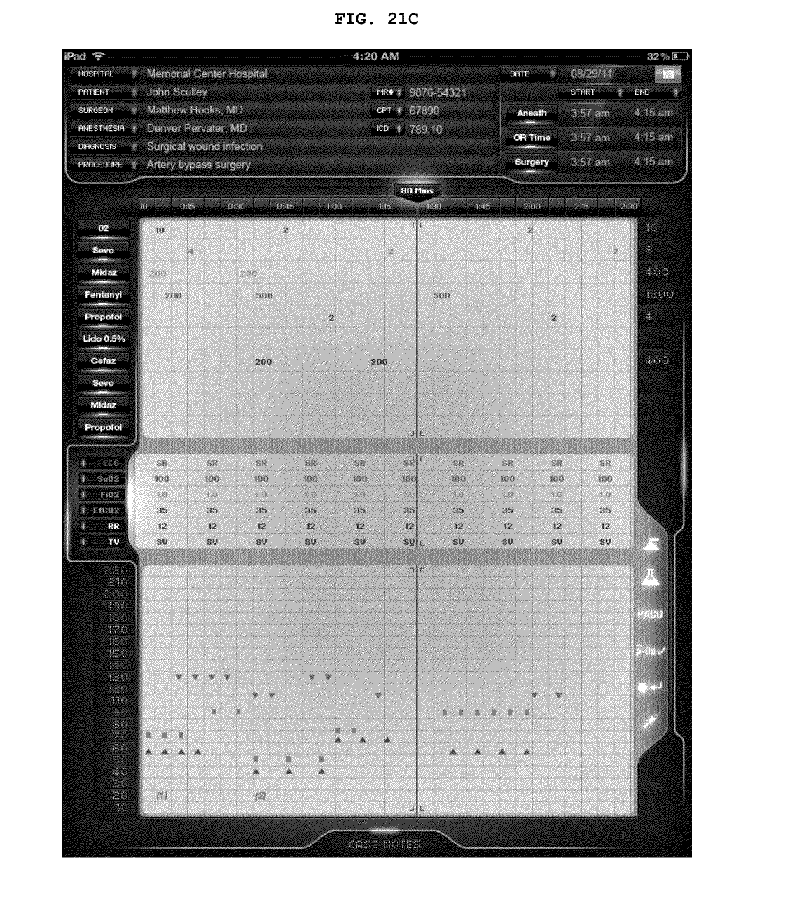

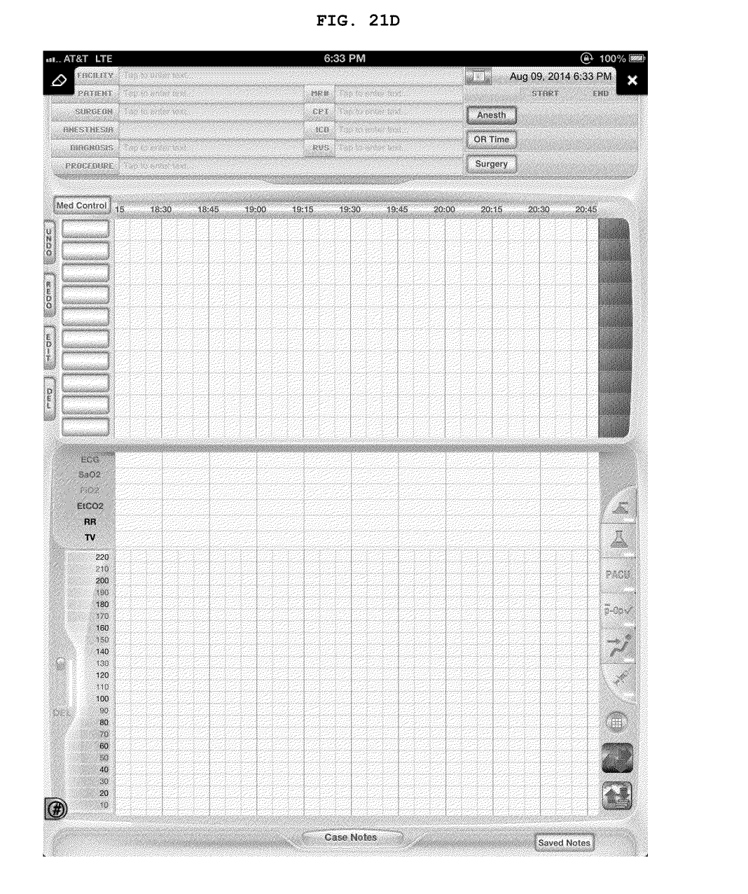

In another embodiment of the invention, the application program operatively enables a means to change the visualization of the invention on a multi-function gesture-sensitive device. This embodiment enables an anesthesiologist to better see the application in different lighting environments frequently encountered in operating room settings. There is also the possibility that the anesthesiologist is wholly or partly color blind requiring this embodiment to best use the application. The application program accomplishes this through a combination of techniques comprising, changing the color palette, the brightness of the gesture-sensitive interface and saturation settings (FIG. 21A-FIG. 21D).

What is claimed is:

1. A graphical user interface produced by an application program, comprising:
an application program window being generated by the application program on a computing device having a gesture-sensitive interface, the application program window enabling a user to manage blood pressure vital sign content in an electronic anesthesia record, the application window comprising a first sub-view, second sub-view, third sub-view, forth sub-view and fifth sub-view;
wherein the first sub-view displays vertical scale of integers being dually purposed to represent blood pressure and heart rate values from low integers on the bottom to high integers on the top;
wherein the second sub-view displays, to the right of the first sub-view, at least a portion of a grid for managing blood pressure vital sign content, the grid containing a plurality of rows and a plurality of columns, the rows representing distinct blood pressure or heart rate values, the columns representing time advancing forward from left to right, such that a value recorded on the grid represents blood pressure vital sign content based on time, the rows being aligned horizontally to the integers displayed in the first sub-view;
wherein the third sub-view displays a timeline of the electronic anesthesia record based on time of day, such that time progresses from left to right, the timeline comprising a plurality of time increments, each increment being aligned to a column of the second sub-view;
wherein the third sub-view displays a plurality of time-based events being tracked by the application program related to the electronic anesthesia record;
wherein the fourth sub-view displays information in real-time under gesture by the user in the second sub-view, such that the application program displays the fourth sub-view above all other user interface controls at the initiation of a gesture and hides the fourth sub-view upon completion of the gesture;
wherein the fifth sub-view displays an apparatus for configuring display characteristics of blood pressure content.

2. The graphical user interface as recited in claim 1, wherein the application program window further contains one user interface control per blood pressure to permit the user the ability to switch the focus between blood pressures under management.

3. The graphical user interface as recited in claim 2, wherein the user interface control is movable via a gesture.

4. The graphical user interface as recited in claim 1, wherein the application program window further contains a user interface control configured to permit the user to undo previous actions.

5. The graphical user interface as recited in claim 1, wherein the application program window further contains a user interface control configured to permit the user to redo previous actions.

6. The graphical user interface as recited in claim 1, wherein the application program window further contains a user interface control configured to permit the user to edit blood pressure vital sign content via a gesture based means.

7. The graphical user interface as recited in claim 1, wherein the application program window further contains a user interface control configured to permit the user to delete blood pressure vital sign content via a gesture based means.

8. The graphical user interface as recited in claim 1, wherein the application program window further contains a user interface control configured to permit the user to turn on and off a snap-to-grid enablement via a gesture based means.

9. The graphical user interface as recited in claim 1, wherein the application program window further contains a user interface control configured to permit the user to add case notes to the second sub-view via a gesture based means.

10. The graphical user interface as recited in claim 1, wherein the application program window further contains a user interface control configured to permit the user to display the fifth sub-view via a gesture based means.

11. An apparatus for managing blood pressure vital sign content in an electronic anesthesia record, comprising:
means for displaying vertical scale of integers being dually purposed to represent blood pressure and heart rate values;
means for displaying at least a portion of a grid for managing blood pressure vital sign content;
means for displaying a timeline of the electronic anesthesia record based on time of day;
means for tracking at least one time-based event associated with the electronic anesthesia record;
means for displaying information in real-time under gesture;
means for configuring blood pressures to be managed.

12. The apparatus as recited in claim 11, wherein the apparatus further includes a means for undoing previous actions via gesture based means.

13. The apparatus as recited in claim 11, wherein the apparatus further includes a means for redoing previous actions via gesture based means.

14. The apparatus as recited in claim 11, wherein the apparatus further includes a means for adding one blood pressure value at a time via gesture based means.

15. The apparatus as recited in claim 11, wherein the apparatus further includes a means for adding blood pressure value in mass via gesture based means.

16. The apparatus as recited in claim 11, wherein the apparatus further includes a means for editing blood pressure values in mass via gesture based means.

17. The apparatus as recited in claim 11, wherein the apparatus further includes a means for deleting at least one blood pressure value via gesture based means.

18. The apparatus as recited in claim 11, wherein the apparatus further includes a means for inspecting at least one blood pressure value via gesture based means.

19. The apparatus as recited in claim 11, wherein the apparatus further includes a means for scale-shifting via gesture based means.

20. The apparatus as recited in claim 11, wherein the apparatus further includes a means for time shifting via gesture based means.

21. The apparatus as recited in claim 11, wherein the apparatus further includes a means for controlling the placement of blood pressure values with a snap-to-grid function via gesture based means.

22. The apparatus as recited in claim 11, wherein the apparatus further includes a means for zooming and magnifying blood pressure values via gesture based means.

23. The apparatus as recited in claim 11, wherein the apparatus further includes a means for adding case notes via gesture based means.

24. The apparatus as recited in claim 11, wherein the apparatus further includes a means for horizontally scrolling to change the displayed blood pressure vital sign content under management via gesture based means.

25. The apparatus as recited in claim 11, wherein the apparatus further includes a means for changing the visual characteristics of at least a portion of the application program window via gesture based means.

26. A computer-implemented method, performed by an application program on a gesture-sensitive device, for adding blood pressure vital sign content, in mass, in an electronic anesthesia record, via gesture based means, each blood pressure vital sign value comprising a systolic part, a diastolic part and a heart rate part, the method comprising the steps of:
(a) performing a first gesture over the systolic part of a pre-existing blood pressure value;
(b) performing a second gesture, during which, the application program inputs new systolic parts at time based increments along the path of the second gesture;
(c) performing the first gesture over the diastolic part of the pre-existing blood pressure value;
(d) performing the second gesture, during which, the application program inputs new diastolic parts at time based increments along the path of the second gesture;
(e) performing the first gesture over the heart rate part of the pre-existing blood pressure value; and
(f) performing the second gesture, during which, the application program inputs new heart rate parts at time based increments along the path of the second gesture.

27. A computer-implemented method as recited in claim 26, wherein the method further comprises the steps of:
(a) displaying the blood pressure value and time value corresponding to the systolic part of the pre-existing value under the first gesture;
(b) displaying the blood pressure value and time value corresponding to the new systolic value under the second gesture in real time;
(c) displaying the blood pressure value and time value corresponding to the diastolic part of the pre-existing value under the first gesture;
(d) displaying the blood pressure value and time value corresponding to the new diastolic value under the second gesture in real time;
(e) displaying the heart rate value and time value corresponding to the heart rate part of the pre-existing value under the first gesture; and
(f) displaying the heart rate value and time value corresponding to the new heart rate value under the second gesture in real time.

28. A computer-implemented method, performed by an application program and shown within an application program window, on a gesture-sensitive device, for adding an individual blood pressure vital sign value in an electronic anesthesia record, via gesture based means, the blood pressure vital sign value comprising a systolic part, a diastolic part and a heart rate part, the method comprising the steps of:
(a) performing a first gesture to specify the location of the systolic part at or near a desired pressure and time;
(b) displaying the pressure and time of the systolic part corresponding to the position under the first gesture;
(c) performing an optional second gesture to obtain the exact desired pressure and time for the systolic part if the displayed pressure and time requires adjustment;
(d) performing a first gesture to specify the diastolic part on or near a desired pressure, wherein the time value is aligned to the time of the systolic part;
(e) displaying the pressure and time of the diastolic part corresponding to the position under the second gesture;
(f) performing an optional second gesture to obtain the exact desired pressure for the diastolic part if the displayed pressure requires adjustment;
(g) performing a first gesture to specify the heart rate part on or near a desired beats per minute, wherein the time value is aligned to the time of the systolic part and diastolic part;
(h) displaying the beats per minute and time of the heart rate part corresponding to the position under the second gesture, wherein the time is the same as the systolic part and diastolic part; and
(i) performing an optional second gesture to obtain the desired beats per minute for the heart rate part if the displayed heats per minute requires adjustment.

29. A computer-implemented method as recited in claim 28, wherein the method further comprises the step of guiding the placement of the systolic part using a snap-to-grid function, wherein the snap-to-grid function places the systolic part on a uniform time increment, such as one systolic part per minute or one systolic part per five minutes.

30. A computer-implemented method, performed by an application program and shown within an application program window, on a gesture-sensitive device, for editing blood pressure vital sign content in an electronic anesthesia record, via gesture based means, the method comprising the steps of:
(a) performing a first gesture to place the application program into an edit mode;
(b) performing a second gesture, the focus of which forms an initial vertex of a rectangle;
(c) performing a third gesture, wherein the user will move the focus of the third gesture away from the initial vertex and in doing so expanding the area of the rectangle while the application program displays the rectangle in the application program window;
(d) performing a fourth gesture over any portion of the rectangle and moving the rectangle to a new location, wherein upon completion of the fourth gesture, the application program will relocate all bounded blood pressure vital sign content from their original position in the application program window to their new position as determined by the location of the bounding rectangle and their relative location therein;
(e) performing the first gesture a second time to take the application program out of edit mode.

31. A computer-implemented method as recited in claim 30, wherein the method further comprises the step of changing the background color of a portion of the application program window to indicate the application program is in edit mode.

32. A computer-implemented method, performed by an application program and shown within an application program window, on a gesture-sensitive device, for inspecting blood pressure vital sign content in an electronic anesthesia record, via gesture based means, the method comprising the steps of:
(a) performing a first gesture to place the application program into an inspect mode;
(b) performing a second gesture, the focus of which forms an initial vertex of a rectangle;
(c) performing a third gesture, wherein the user will move the focus of the third gesture away from the initial vertex and in doing so expanding the area of the rectangle while the application program displays the rectangle in the application program window, wherein the application program will display additional information about the blood pressure vital sign content bounded by the rectangle, the additional information comprising, the pressure in mmHg if it is a pressure value and the beats per minute if it is a heart rate value;
(d) performing the first gesture a second time to take the application program out of inspect mode.

33. A computer-implemented method, performed by an application program and shown within an application program window, on a gesture-sensitive device, for scale-shifting in an electronic anesthesia record, via gesture based means, the method comprising the steps of:
(a) performing a first gesture on a grid at least a portion of which is displayed in the application program window, the grid having rows representing a scale of blood pressure values from low to high, the grid having a first horizontal region spanning a low range of blood pressure values, a second horizontal region spanning the intermediate range of blood pressure values and a third horizontal region spanning a high range of blood pressure values, the first horizontal region having a first increment between rows, the first gesture causing the application program to change the increment from the first increment to a second increment in the first horizontal region, the second increment being smaller than the first increment, the third horizontal region having a first increment and the application program in response to the first gesture changes the first increment to a second increment, the first increment being smaller than the second increment;
(b) performing the first gesture an optional second time in the first horizontal region wherein the application program changes the increment from the second increment to a third increment such that the third increment is smaller than the second increment.

34. A computer-implemented method as recited in claim 33, wherein the first horizontal region spans the range of 0 mmHg to 49 mmHg.

35. A computer-implemented method as recited in claim 33, wherein the second horizontal region spans the range of 50 mmHg to 150 mmHg.

36. A computer-implemented method as recited in claim 33, wherein the third horizontal region spans the range of 150 mmHg to 220 mmHg.

37. A computer-implemented method as recited in claim 33, wherein the first increment is 10 mmHg per row.

38. A computer-implemented method as recited in claim 33, wherein the second increment is 5 mmHg per row.

39. A computer-implemented method as recited in claim 33, wherein the third increment is 1 mmHg per row.

40. A computer-implemented method as recited in claim 33, wherein the application program changes the background color of the first horizontal region after the first gesture from a first color to a second color.

41. A computer-implemented method, performed by an application program and shown within an application program window, on a gesture-sensitive device, for deleting pre-existing blood pressure vital sign content in an electronic anesthesia record, via gesture based means, the method comprising the steps of:
(a) performing a first gesture to place the application program into a delete mode;
(b) performing a second gesture over pre-existing blood pressure vital sign content and deleting that content as the focus of the second gesture passes over it;
(c) performing the first gesture a second time to take the application program out of delete mode.

42. A computer-implemented method as recited in claim 39, wherein the method further comprises the step of changing the background color of a portion of the application program window to indicate the application program is in delete mode.

43. A computer-implemented method, performed by an application program and shown within an application program window, on a gesture-sensitive device, for time-shifting in an electronic anesthesia record, via gesture based means, the method comprising the step of:
(a) performing a gesture on a grid at least a portion of which is displayed in the application program window, the grid having columns representing increments of time, with time advancing from left to right, such that the application program in response to the gesture, changes the increment between at least two columns from a lower resolution to a higher resolution.

44. A computer-implemented method as recited in claim 43, wherein the method further comprises the step of changing the background color of the columns whose resolution the application program made higher in response to the gesture.

45. A computer-implemented method, performed by an application program and shown within an application program window, on a gesture-sensitive device, for expanding and magnifying at least a portion of the application program window, in an electronic anesthesia record, via gesture based means, the method comprising the step of:
(b) performing a gesture on the application program window, the application program window being divided into at least a first and second area, each first and second area when separately gestured will expand to substantially fill the application program window and the content of the area under gesture will be magnified by a first magnification
(c) performing the gesture an optional second time, in response to which, the application program will increase the magnification of the area under gesture, now substantially filling the application program window, by a second magnification.
